(19) United States
(12) Patent Application Publication
(10) Pub. No.: US 2007/0062865 A1
(43) Pub. Date: Mar. 22, 2007

(54) Novel biological flocculants and production methods

(75) Inventors: Heng-Chuan Wang, Taipei (TW); Ming-Hsin Li, Chungli City (TW); Hsu-Wen Tsang, Taipei (TW); Mei-Mao Wu, Chunghe City (TW); Hsui-Ping Pearl Lin, Tempe, AZ (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Development Center for Biotechnology

(21) Appl. No.: 11/601,693

(22) Filed: Nov. 20, 2006

Related U.S. Application Data
(62) Division of application No. 10/762,429, filed on Jan. 21, 2004.
(60) Provisional application No. 60/442,581, filed on Jan. 22, 2003.

(51) Int. Cl. B01D 69/02; C12N 1/20

(52) U.S. Cl. 210/601; 435/252.31

(57) ABSTRACT

The present invention relates to a novel flocculant comprising bacteria of the Bacillus genus cultivated in a culture medium comprising soybean protein, glucose, molasses, and yeast. Alternatively, the flocculant comprises soybean protein. The present invention also relates to methods of making the flocculant, including fermentation method for mass production, and methods of using the flocculant in water treatment.

### DESCRIPTION OF THE INVENTION

1. Field of the Invention

This invention relates generally to novel flocculants for water treatment and more particularly to flocculants comprising bacteria of the genus Bacillus and the appropriate culture medium composition and production methods thereof.

2. Background of the Invention

Flocculation agents including coagulants and flocculants mainly act to destabilize the colloids and suspended particles in the liquids, thus making the colloids and suspended particles agglomerate into “flocs” with larger sizes and faster settling speeds. For water treatment or wastewater treatments, the coagulation unit involves coagulation treatment and flocculation treatment. At first, a coagulant is quickly mixed with the water or wastewater to disrupt the charge balance of the colloids and suspended particles. Thereafter, a flocculant is added and the mixture is stirred slowly, so as to increase the incidence and efficiency of collision between the particles and thereby facilitate the formation of larger and heavier floc with firm structures from the suspended particles. The flocs will be removed in the subsequent treatment units such as sedimentation or flotation, thus achieving the goal of separating the solids from the liquids in the water or wastewater treatment.

In general, flocculants can be categorized based on their main components as one of three main types: (1) chemical inorganic salt coagulants, such as ferric sulfate, aluminum sulfate, and aluminum chloride etc., (2) chemical synthetic polymeric flocculants, including polyacrylamide, polyacrylic acid and polyethylene imine, (3) natural biological polymers, including chitosan, algin, poly-y-glutamic acid and extra cellular biopolymer (Mortimer D. A. (1991) Polymer International 25(1): 29-41; Rosenberg E. (1986) Critical Reviews in Biotechnology 3(2):109-132; Shih I. L. & Van Y. T. (2001) Bioresource Technology 79(3):201-115; Yokoi H. M. et al. (1996) Biological Techniques 10(10):789-792; Cardenas G. et al. (2001) International Journal of Biological Macromolecules 28:167-174).

The inorganic salt coagulants, having advantages of low-cost, good coagulation activity and broad application, have widely been applied in industry wastewater treatments. However, the application of inorganic salt coagulants has serious disadvantages, including the production of large amount of sludge and failure to remove sludge and heavy metal remnants, thus leading to the extensive applications of organic chemical Coagulants in treating water in recent years. Although the costs of the chemical organic polyelectrolytes are high, the chemical organic polyelectrolytes provide high agglutination, fast sedimentation rates, less sludge production and good dewatering ability of sludge. So far, the water treatment plants employ the chemical organic polyelectrolytes together with the chemical inorganic salt coagulants. However, the monomers of the chemical organic polymeric flocculant are neurotoxins or carcinogens, which are hazardous to both the plant workers and the environment.

Indeed, in recent years, many reports have indicated the negative influences of environmental hormones on human beings and the ecosystem, which draws the attention of countries around the world to the consequences of minor organic materials in water. Therefore, the control of organic material traces in the water will be the focus of future environmental regulations. Because biological flocculants are biologically nontoxic and are biologically degradable, their application will not induce negative effects to the current treatment systems and have great potential in replacing the market of organic polyelectrolytes. Since the 1980s, countries at the forefront of environmental protection have actively developed biological flocculants. It has been found that microbes, including yeast, fungi and bacteria, have capability of generating metabolite products with flocculating activity. For example, Rhodococcus erythropolis and Nocardia amarae produce protein flocculants, and Arathrobacter sp. and Aarcuadendron sp. TS-4 produce glycoprotein flocculants. However, because the fermentation and product recovery costs of the biological flocculants are high, the commercialized products of the biological flocculants are usually available for the food industry or the cosmetic industry, rather than for industrial wastewater treatment (Shih I. L. & Van Y. T. (2001) Bioresource technology 2001.79(3):207-115; Kurane R. (1997) Environmentally Friendly Products and Processes for the 21^{st} Century, in Global Environmental Biotechnology 759-769; Ganjidoust H. et al. (1997) Water Science and Technology 35(2-3):291-296; Gassenschmidt U. et al. (1995) Biochimica et Biophysica Acta 1243:447-481; Kurane R. et al. (1994) Bioscience, Biotechnology and Biochemistry 58(2): 428-429; Kurane R. et al. (1994) Bioscience, Biotechnology and Biochemistry 58(11) 1977-1982; Kurane R. & Matsuyama H. (1994) Bioscience, Biotechnology and Biochemistry 58(9) 1589-1594; Salehizadeh H. et al. (2000) Biochemical Engineering Journal 5:39-44; Takeda M. et al. (1992) Journal of Fermentation and Bioengineering 74(6): 408-409; Thompson I. M. & Forster C. F. (1983) Biotechnology Letters 5(11) 761-766; Wang Z. et al. (1994) Biotechnology Techniques 8(11): 831-836). The existing commercialized products of the biological flocculants for the water treatment are few, including microbial products in the name of BIOFLOC from Bio R&Ds Co., Ltd. of Korea and a polymer of cellulose and sulfates in the name of BIO-FLOCK from OCETA Inc. of Canada. Nevertheless, compared with the chemical flocculants, these biological flocculants have lower efficiency and higher costs.

Therefore, there is the need to improve biological flocculants to achieve at least the same efficiency as chemical flocculants and to also reduce biological flocculants' production cost.

### SUMMARY OF THE INVENTION

The present invention addresses these problems by providing a flocculant that utilizes the flocculating capability of a strain of the Bacillus sp. bacteria. The invention also provides for a flocculant that is based on a culture medium.

In one embodiment of the invention, there is a strain of the Bacillus sp. bacteria which has shown flocculating activity towards kaolin suspensions or industrial wastewater.

The invention also provides flocculants that comprise the metabolites generated by a strain of the Bacillus sp. bacteria or, in the alternative, the flocculant comprises the bacterial strain of Bacillus sp. and the appropriate culture medium comprising soybean protein, glucose, molasses, and yeast. The flocculant may also comprise culture medium comprising soybean protein. Flocculants of the present invention have the same flocculating effect as chemical flocculants and the production cost is acceptable given the low raw material cost and high bacteria recovery rate.

In another embodiment of the invention, the process for making the flocculant comprises incubating the Bacillus sp. bacteria for 60 to 108 hours. In yet another embodiment, the process further includes the step of treating said bacteria under high temperature and or high pressure. The invention also includes a process for making powdered flocculant from the Bacillus sp. bacteria precipitates by spray drying.

The present invention further provides for a method of treating water that comprises the application of said powdered flocculant or soybean protein.

It is to be understood that both the foregoing general description and the following detailed description are exemplary and explanatory only and are not restrictive of the invention, as claimed.

The accompanying drawings, which are incorporated in and constitute a part of this specification, illustrate one (several) embodiment(s) of the invention and together with the description, serve to explain the principles of the invention.

### BRIEF DESCRIPTION OF THE DRAWINGS

FIG. 1 is a flow chart of fermentation cultivation.

FIG. 2 is a diagram showing the influence of the settling time upon the absorbance of the supernatant. The bacterial solution used in this flocculation experiment is produced from the fermentation culture medium GSM, while the testing solution of the flocculation is the textile dyeing wastewater (Shin-Long).

FIG. 3 is a diagram showing the influence of the fermentation time upon the flocculating activities of the bacterial solutions. The bacterial solution used in this flocculation experiment is produced from the fermentation culture medium GSM, while the testing solution of the flocculation is the textile dyeing wastewater (Shin-Long).

FIG. 4 is a diagram depicting comparison of the glucose metabolic rates for the culture media GSM and GSMY during fermentation processes.

FIG. 5 is a diagram depicting comparison of the total bacteria count for the culture media GSM and GSMY during fermentation processes.

FIG. 6 is a diagram depicting comparison of the flocculating activities by the fermentation solution and the organic chemical flocculants. The testing solution is obtained by adding 500 mg/L aluminum chloride solution into the textile dyeing wastewater (Shin-Long) and adjusting the pH value to 6.57.

FIG. 7 is a diagram showing the influence of thermal treatment on the flocculating activities of the production formulations.

FIG. 8 is a diagram depicting biological toxicity of the biological flocculant. In general, “50% of effects” represents that the luminescent intensity of the luminescent microbe in the sample is reduced to half, compared with no reduction at all. From the test results, when the concentration of the biological flocculant is as high as 1% (10000 mg/L), the reduction of the luminescent intensity is less than 50%. This means that the biological flocculant has no MicroTox® biological toxicity under common concentrations (20-100 mg/L)

FIG. 9 is a diagram depicting changes in biological toxicity of the treated textile dyeing wastewater. The testing solution is prepared by adding 350 mg/L PACl into the textile dyeing wastewater (Shin-Long), and then adjusting the pH value to 6.3 using 3 N NaOH solution. Labels: raw water=the textile dyeing wastewater (Shin-Long); With PACl=testing solution control; With PACl+EA-630=testing solution added with 2 mg/L EA-630; With PACl+BioFloc B=testing solution added with 0.2 mg/L BioFloc B

FIG. 10 is a diagram depicting the influence. of the dosage of the biological flocculant on the settled sludge volume. The biological flocculant BioFloc A is the fermentation solution of the microbe B. endophyticus; BioFloc B is the culture medium prepared under high temperature and high pressure (121° C., 1.5 atm). The testing solution is prepared by adding 350 mg/L PACl into the textile dyeing wastewater (Shin-Long), and then adjusting the pH value to 6.3 using 3 N NaOH solution. This is the control. For the supernatants with similar turbidity, as the settled sludge volume (SSV) is smaller, the sludge settling speed is faster and the sludge concentration is higher.

FIG. 11 is a diagram showing the comparison of the flocculating activities by the biological flocculant BIOFLOC B and the organic chemical flocculants The testing solution is prepared by adding 350 mg/L PACl into the textile dyeing wastewater (Shin-Long), and then adjusting the pH value to 6.52 using 3 N NaOH solution. This is the control. For the supernatants with similar turbidity, as the settled sludge volume (SSV) is smaller, the sludge settling speed is faster and the sludge concentration is higher.

### DETAILED DESCRIPTION OF THE INVENTION

The present invention provides for strains of bacteria having flocculating capability that can be used in water treatment. The invention further provides for flocculants comprising the bacteria and/or their metabolite products. In another embodiment of the invention, the flocculant comprises culture medium soybean protein. The invention is also directed to processes for making the flocculants and method of using the flocculants to treat water.

Definitions

As used herein, the term “flocculation agent” means coagulant and/or flocculent or a combination of coagulant(s) and flocculant(s).

As used herein, the term “flocculating effect” means the ability to increase the settling rate of suspended particles and to increase the volume of settled sludge as compared to an isolated aqueous suspension without any substance added.

As used herein, the term “kaolin suspensions' refers to aqueous suspension comprising kaolin particles.

As used herein, the term “industrial wastewater” refers to wastewater from any industrial plant, including but not limited to industrial plants in the dyeing, cosmetic and food industries.

As used herein, the term “water” refers to any water to be treated by flocculent which includes but is not limited to industrial wastewater.

As used herein, the term “tap water” refers to water for mixing with flocculants which can be water from the tap, distilled water, sterile water or any water medium for carrying the flocculants.

As used herein, the term “bacterial solution” refers to a solution containing nutrient broth for bacteria growth and the bacteria themselves.

As used herein, the term “nutrient broth” refers to solution containing substances required for the microorganisms for its growth and sustainment of life.

As used herein, the term “culture. medium” refers to a medium containing the necessary substances for the growth and sustainment of the microorganisms' life.

As used herein, the term “carbon source” includes but is not limited to corn starch, glucose, sucrose, and sugar molasses etc.

As used herein, the term “nitrogen source” includes but is not limited to peptone, hydrolytic soybean protein, soybean protein MP-90, soybean protein Supro-620, soybean protein EG-90, soybean protein HI-90, yeast extract, ammonium sulfate, and ammonium chloride etc.

As used herein, the terms shaking” and “shaken” etc. refer to being shaked in a shaking incubator, and the shaking can take place in any direction, i.e. horizontal, vertical, orbital etc.

As used herein, the term “high temperature” refers to above 37° C.

As used herein, the term. “thermal treatment” refers to treatment under high temperature.

As used herein, the term “high pressure” refers to above atmospheric pressure of 1 atm.

As used herein, the term “collect”, “collecting” or “collection” refers to gathering generally homogenous components in a sample by any manner including but not limited to centrifuge or filter.

As used herein, the term “precipitates” refers to bacterial cell precipitates, metabolite products of bacterial cells or both the bacterial cell precipitates and their metabolites separated from the solution or suspension.

As used herein, the terms “metabolite”, “metabolites”, and “metabolite products” may be used interchangeably and they all refer to products of metabolism.

As used herein, the terms “spray drying” and “spray dried” etc. refer to the use of sprayer dryer to increase the solid content of the bacterial solution.

As used herein, the term “fermented solution” refers to solution being subjected to fermentation.

As used herein, the term “activating” refers to boosting cultivation of bacterial strains that were frozen and preserved such that the bacterial can serve as seed bacteria for main production.

Bacterial Strain with Flocculating Capability

The present invention provides for, in a first aspect, a biological flocciulant comprising a bacterial strain with flocculating capability. This strain of bacteria is in the Bacillus genus and displays flocculating effect towards kaolin suspensions or, in the alternative, industrial wastewater. Kaolin suspensions are aqueous suspensions comprising kaolin particles, and industrial wastewater may be wastewater coming from any industrial plant, including but not limited to industrial plants in the dyeing, cosmetic, and or food industries. Screening for bacteria with the flocculating capability can be carried out by detecting the flocculating behavior displayed by the solutions containing the microbes, i.e. bacterial solutions, by eye-measuring the turbidity differences between the supernatants of the experimental solutions and the controls, specifically by taking note of the settling rate of the suspended particles, the absorbance of the supernatant, and the volume of the settled sludge. The experimental solutions would be kaolin suspensions or industrial wastewater added with designated amount of bacterial solutions, and the controls would be kaolin suspensions or industrial wastewater without the bacterial solution. The flocculating capability of the selected bacteria can be used for treating wastewater or for any other water treatment purposes.

In one embodiment of the invention, the bacterial strain displaying the flocculating effect is of the species Bacillus endophyticus. In another embodiment of the invention, the flocculating bacteria is of the species Bacillus cereus. In yet another embodiment, the flocculating bacteria is of the species Bacillus subtilis

In a more particular embodiment of the present invention, the bacterial strain of the genus Bacillus displays flocculating effect towards kaolin suspensions that comprises kaolin and CaCl₂.2H₂O. Alternatively, flocculating effect is displayed towards kaolin suspensions that comprises about 1.25% kaolin and about 3% CaCl₂.2H₂O. According to one specific embodiment, the bacterial strain displays flocculating effect towards kaolin suspensions, which comprises about 20 ml of about 1.35% kaolin and about 0.5 ml of about 3% CaCl₂.2H₂O. In addition, the bacterial solution containing the Bacillus bacteria is incubated under 30° C. in a shaking incubator for about 15 hours. Shaking is taken place in a shaking incubator and the direction of shaking is not limited i.e. it can be horizontal, vertical, orbital etc.

The invention further provides for a bacterial strain of the Bacillus genus that retained its flocculating capability even after high temperature treatment. Biological flocculent containing the microbes are recovered by spray drying, carried out under high temperature, in the production process. Thus, flocculating bacterial strains that can withstand high temperature treatment is desirable. Screening for such bacteria is carried out by high temperature treatment followed by a test for any change in the flocculating capability. High temperature treatment can be performed by heating the flask containing the bacterial solutions or by any other manner known in the art. Temperature above the normal room temperature of 37° C. would be considered high temperature. The test for the flocculating capability can be performed with the bacterial solution or with the bacterial cell precipitate dilution obtained after the high temperature treatment.

In one embodiment, the invention includes a bacterial strain that still displays flocculating effect after undergoing high temperature treatment at about 60° C. In another embodiment, the bacterial strain retains the flocculating effect towards kaolin suspensions or industrial wastewater after high temperature and high pressure treatment. More specifically, the high temperature and high pressure treatment is carried out at 122° C. and 1.5 atm respectively. In a specific embodiment, the invention provides for a bacterial strain that displays flocculating effect after undergoing high temperature treatment at 122° C. and high pressure treatment at 1.5 atm. in a bacterial solution comprising about 100 ml of nutrient broth and is incubated under about 30° C. and shaking for about 48 hours.

Culture Composition

The present invention is also directed to certain culture compositions that display the flocculating behavior, and the culture comprises Bacillus endophyticus, soybean protein, glucose, and molasses. Any hydrolytic soybean protein may be used. In the alternative, other nitrogen sources such as peptone, yeast extracts and inorganic nitrogen sources, e.g., ammonium sulfate and ammonium chloride etc., may also be used. Glucose is the carbon source for the bacteria, and other carbon sources include corn starch, sucrose, and sugar molasses. Molasses is added to provide for the microelements of calcium, sodium, sulfur, iron, zinc, manganese, cobalt, and copper etc. These microelements may be added separately or provided in other forms.

In a more particular embodiment, the soybean protein MP-90 is about 15 grams/liter. In yet another embodiment, the soybean protein MP-90 is about 6 to 24 grams/liter, glucose is about 15 grams/liter, and molasses is about 2 grams/liter. The invention also includes a culture comprising the bacterial strain Bacillus endophyticus, soybean protein Supro-620, glucose, and molasses. In another embodiment, the amount of soybean protein Supro-620 is about 15 grams/liter. In yet another embodiment, the amount of soybean protein Supro-620 is about 6 to 30 grams/liter, glucose is about 10 to 20 grams/liter, and molasses is about 2 to 5 grams/liter.

The invention is further directed to a culture comprising the bacterial strain Bacillus cereus, soybean protein Supro-620, glucose, and molasses. In one embodiment, the amount of soybean protein Supro-620 is about 15 grams/liter. The culture medium can further comprise about 10 to 15 grams/liter of glucose and about 2 to 10 grams/liter of molasses. Different carbon and nitrogen sources may be substituted. In another embodiment, the culture comprises the bacterial strain of Bacillus cereus, about 15 grams/liter of soybean protein Supro-620, about 10 to 20 grams/liter of corn starch, and about 5 to 10 grams/liter of molasses.

The invention further provides for a culture comprising a bacterial strain of the Bacillus genus and lactic fermentation waste.

Process for Making Biological Flocculant

In another aspect of the invention, the invention provides a process for making a biological flocculant that comprises the steps of inoculating the bacterial strain of the Bacillus genus onto a culture medium that contains the necessary substances for the growth of the bacteria, incubating it for about 60 to 108 hours (a suitable culture time determined by experiments), treating the culture under high temperature and high pressure, and collecting the precipitates. The precipitates may be bacterial cell precipitates along with the metabolites, or in the alternative, the precipitates may be just the metabolite products separated from the bacterial cells by centrifuge or filtering process or any other collecting method. Subjecting the flocculent to high temperature treatment enhances the flocculating effect. by increasing particle settling rate.

In a more particular embodiment of the invention, the bacterial strain used is of Bacillus endophyticus which is inoculated onto a culture medium and incubated for about 60 to 108 hours. The culture is then treated to high temperature and high pressure followed by the collection of precipitates. In another embodiment of the invention, the process for making biological flocculant comprises the steps of inoculating the bacterial strain of the Bacillus genus onto a culture medium, incubating it for about 60 to 108 hours, and collecting precipitates without any prior thermal treatment. In a more particular embodiment, the culture is incubated for about 70 to 80 hours or in the alternative for about 90 to 100 hours.

The invention provides for another process of making biological flocculant by incubating bacterial strain of the Bacillus genus in a culture medium for about 70 to 100 hours, to obtain a bacterial solution with flocculating capability.

Flocculant can come in liquid or powder form. Powdered flocculant has longer shelf life. The present invention further provides for a process for making powdered biological flocculant comprising the steps of inoculating the bacterial strain of the Bacillus genus onto a culture medium, incubating the inoculated culture medium for about 72 to 96 hours and spray drying the culture medium with a spray dryer to convert it into powdered form. The culture medium used should contain at least carbon and nitrogen sources as described above. The extent of spray drying is controlled by monitoring the solid content in the bacterial solutions, for example, to spray dry until the solid content of the bacterial solutions reaches 80-90%. In a more particular embodiment of the invention, the spray dryer is set with an inlet temperature of about 110° C. and an outlet temperature of about 90° C. In another embodiment of the invention, the process for making powdered biological flocculant comprises the steps of inoculating the bacterial strain of Bacillus endophyticus onto a culture medium, incubating the culture medium for about 72 to 96 hours and spray drying the culture medium. More particularly, the culture medium comprises soybean protein, glucose, and molasses. In the alternative, more specifically, the culture medium comprises about 15 grams/liter of soybean protein, about 10 grams/liter of glucose and about 5 grams/liter of molasses. In yet another embodiment of the invention, the process for making powdered biological flocculant comprises the steps of inoculating bacterial strain of Bacillus cereus onto a culture medium, incubating the inoculated culture medium for about 72 to 96 hours and then spray drying the culture medium. In a further embodiment, the culture medium comprises soybean protein and cornstarch. Specifically, the culture medium comprises about 25 grams/liter of soybean protein and about 10 grams/liter of corn starch.

Novel Flocculant

The present invention also provides for a flocculant comprising a fermented solution, a solution being subjected to fermentation, comprising bacterial strain of the Bacillus genus, glucose, soybean protein, molasses, and yeast.

The invention also provides for a flocculant comprising precipitates collected from culture medium comprising bacterial strain of Bacillus endophyticus, soybean protein, glucose, and molasses.

In another embodiment, the flocculant comprises the strain of Bacillus endophyticus, soybean protein, glucose, and molasses. Furthermore, it is incubated for about 70 to 100 hours and undergoes spray drying treatment.

In yet another embodiment, the invention provides for a flocculant comprising bacterial strain of the Bacillus genus, about 50 grams/liter to 100 grams/liter of nitrogen sources, molasses, and yeast. More particularly, the nitrogen source is soybean protein.

The invention further provides for a flocculant comprising about 15to 120 grams/liter of soybean protein. More particularly, the soybean protein is subjected to thermal treatment. The thermal treatment may be carried out at 121° C., or alternatively, at 121° C. and 1.5 atm. The thermal treatment lasts about twenty minutes.

In another embodiment, the invention provides for a flocculation agent comprising one of the various flocculants described above and ferric chloride, a coagulant, or in the alternative, another coagulant, aluminum chloride may be used. These two coagulants are found to improve the flocculating effect.

Alternative carbon and nitrogen sources may be used for glucose and soybean protein, respectively, in the culture medium for the flocculants.

Bacterial Culture Fermentation Process

The present invention is also directed to a bacterial culture fermentation process comprising the steps of preparing a culture medium comprising bacterial strain of the Bacillus genus, glucose, soybean protein, molasses, and yeast, fermenting the culture for about 40 to 48 hours, setting ventilation volume to about 0.5 to 1.0 VVM, and adjusting initial pH of the production culture medium to about 6-7. The fermentation process tries to establish economical production methods for the biological flocculants by designing the appropriate culture medium, the most suitable conditions for fermentation and the fermentation strategy. The main purpose is to achieve large bacterial cell production, short fermentation time, high activity products and low production costs.

Water Treatment Method

The present invention further provides for a method of treating water comprising the steps of introducing into water to be treated the flocculant comprising the fermented solution of bacterial strain of the Bacillus genus, glucose, soybean protein, molasses, and yeast. More particularly, the dosage of the flocculant added is 2 ml/liter.

The present invention is also directed to another method of treating water comprising the steps of mixing powders of biological flocculant with water to form a 1% powder solution. The flocculant is derived from medium comprising bacterial strain Bacillus endophyticus, soybean protein, glucose, and molasses, wherein the culture medium is incubated for about 70 to 1.00 hours and subjected to spray drying treatment. Then, the pH of the 500 ml of water to be treated is adjusted followed by the addition of about 1 ml of the 1% powder solution into the 500 ml water to be treated. In a more particular embodiment, the pH ranges from about 6.3 to 6.5 for the water to be treated. In addition, the pH may be adjusted by adding poly aluminum chloride or other appropriate agent. Also, water to be treated can be wastewater from plants in the food industry.

The invention also provides for another method of water treatment comprising the step of adding soybean protein wherein the concentration of the soybean protein is about 15 to 120 grams/liter. In a more particular embodiment, the soybean is introduced in a dosage of about 0.2 ml/liter to 0.5 ml/liter. Alternatively, the method of water treatment further comprises the step of subjecting the 100 grams/liter of soybean protein under thermal treatment of 121° C. at 1.5 atm. for 20 minutes.

Method of Activating Preserved Bacteria

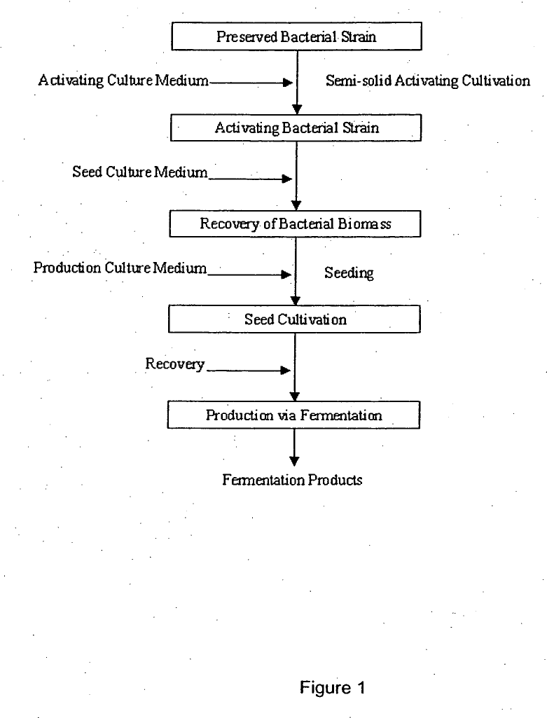

The invention also provides for a process for activating a preserved bacterial strain. The activation step is part of the fermentation-style production technology for scaling up the biological flocculant comprising bacteria such as from a 5 liter fermentation tank to a 150 liter fermentation tank. FIG. 1 is a detailed flow chart for the development strategy of the fermentation of the biological flocculant comprising bacteria. The fermentation production procedure includes the activation of the preserved flocculant bacterial strains, preparation of the seed bacteria culture, and scaling up from fermentation tanks in various stages to production in the massive main fermentation tank.

In the present invention, the bacterial strain is activated by administering a culture medium for boosting cultivation of bacterial strains that were frozen and preserved such that the bacteria can serve as seed bacteria for production. The process for activating preserved bacterial strain comprises the steps of streaking the preserved bacterial strain of Bacillus endophyticus onto a tryptic soy agar with a concentration of about 40 grams/liter and incubating the culture medium for about 1 to 2 days under about 30° C.

Both the foregoing description and the following detailed account are exemplary and explanatory only and are not restrictive of the invention, as claimed. Moreover, the invention is not limited to the particular embodiment described, as such may, of course, vary. Further, the terminology used to describe particular embodiments is not intended to be limiting, since the scope of the present invention will be limited only by its claims.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

The following examples further illustrate the invention. They are merely illustrative of the invention and disclose various beneficial properties of certain embodiments of the invention. The following examples should not be construed as limiting the invention.

### EXAMPLES

The present invention is further illustrated by the following examples, which should not be construed as limiting in any way.

The practice of the present invention will employ, unless otherwise indicated, conventional techniques of cell biology, cell culture, fermentation, and water treatment, which are within the skill of the art. Such techniques are explained fully in the literature.

The following examples illustrate the production of biological flocculants through fermentation employing novel microbial strains and the use of the biological flocculants thus produced in the treatment of wastewater from various industries.

### Example 1

### Preliminary Screening of Microbial Strains

Screening was carried out with respect to microbes preserved in the Development Center for Biotechnology (hereinafter referred to as the “Center”) to pick out microbes that produce biological flocculants. The biological flocculants comprising microbes can be detected by the flocculating behavior displayed by the solutions containing the microbes. Therefore, bacterial strains having the potential of producing biological flocculants were primarily screened by examining the flocculating activities of the microbial solutions, containing the same fixed culture medium, toward the standard kaolin suspensions, which was analyzed by eye-measuring the turbidity differences between the supernatants of the experimental solutions (kaolin suspensions added with designated amounts of bacterial solutions) and the controls (kaolin suspensions without the bacterial solution). The bacterial solutions having flocculating activities toward the kaolin suspensions were tested again against the industrial wastewater. Microbes with flocculating activities toward the industrial wastewater were chosen as possible biological flocculants producing microbial strains.

More specifically, first, all 600 and more bacterial strains preserved in the Center were separately incubated with 30 ml Nutrient Broth (Merck) in 125 ml flasks, under 30° C., shaking in 200 rpm, for 15 hours. Second, 30 ml of the bacterial solution and 0.5 ml of 3% CaCl₂.2H₂O solution were added into 20 ml of 1.25% kaolin suspension (kaolin, Riedel-deHaen). Then, the flocculating activity of the bacterial solution was analyzed by eye-measuring the turbidity differences between the supernatants of the kaolin suspensions with the bacterial solutions and without the bacterial solution.

The flocculating capabilities, based on the eye measurement, can be categorized as four grades: 0, 1, 2 and 3. “0” represents no obvious facilitation (acceleration) for the settlement of the suspended particles, while “3” represents the increased settling rate for the particles and having the best flocculating effect. Some bacterial strains displayed the best flocculating effect characterized as “3”, see Table 1 below.

TABLE 1
Comparison of the flocculating efficiency, toward the kaolin suspension, of
the bacterial solutions of 600 and more strains in the Center

| Bacterial | Flocculating | Bacterial | Flocculating | Bacterial | Flocculating | Bacterial | Flocculating |
|---|---|---|---|---|---|---|---|
| strain | effect | strain | effect | strain | effect | strain | effect |
| Y0626 | 2 | K2075-2 | 0 | BS03023-1 | 0 | MH003 | 2 |
| Y1201 | 0 | K3165 | 0 | BS03023-2 | 0 | MH005 | 0 |
| Y1310 | 2 | K3365 | 1 | BS03026 | 0 | BTHP-12 | 3 |
| Y1314 | 2 | K3369 | 0 | BS03027 | 2 | BTHP-11 | 2 |
| Y1316 | 2 | K3462 | 1 | BS03029 | 0 | BTHP-8 | 2 |
| Y1335 | 1 | Y2023 | 0 | BS03031 | 0 | BTHP-7 | 2 |
| Y2105-1 | 3 | Y3104 | 1 | BS03032 | 2 | BTHP-6 | 3 |
| Y2111 | 0 | Y3121 | 2 | BS03033 | 2 | BTHP-13 | 3 |
| Y2113 | 0 | Y3123 | 0 | BS03034 | 0 | BTHP-5 | 3 |
| Y2115 | 1 | Y3302-2 | 0 | BS03037 | 0 | BTHP-4 | 3 |
| K0667 | 3 | Y3307-2 | 1 | BS03042 | 0 | BTHP-3 | 3 |
| K1265 | 2 | Y3314 | 0 | BS03046 | 0 | BTHP-2 | 3 |
| K1277 | 2 | Y3315 | 0 | BS03047-1 | 2 | BTHP-1 | 1 |
| K1963 | 2 | Y3316 | 1 | BS03047-2 | 2 | MH024 | 2 |
| K1966 | 2 | Y3317 | 0 | BS03051 | 1 | MH020 | 2 |
| K2173 | 2 | Y3318 | 0 | BS03053 | 1 | MH016 | 2 |
| K2183 | 2 | Y3319 | 0 | BS03067 | 1 | MH021 | 2 |
| K2184 | 2 | Y3320 | 1 | BS03069 | 1 | MH014 | 2 |
| Y1915 | 1 | K3463 | 3 | BS03070 | 3 | MH011 | 2 |
| Y1931 | 2 | K3465 | 1 | BS04017 | 2 | MH007 | 2 |
| Y1938 | 1 | Y3403-1 | 3 | BS04029-2 | 1 | BS02023 | 2 |
| Y1939-1 | 1 | K3467 | 1 | BS04029-3 | 0 | BS02024-1 | 2 |
| Y1940 | 1 | K3469 | 0 | BS04041 | 1 | BS02024-2 | 2 |
| Y1941 | 3 | K3470 | 0 | BS04045 | 2 | BS02027-1 | 0 |
| Y1944 | 1 | Y3404 | 3 | BS04046 | 0 | BS02027-2 | 0 |
| Y1945 | 2 | Y3406 | 3 | BS04047 | 0 | BS02029 | 2 |
| Y1947 | 2 | K3561 | 0 | BS04057 | 3 | BS02031-1 | 0 |
| Y1952 | 2 | K3563 | 2 | BS04059 | 3 | BS02034 | 0 |
| Y1953 | 2 | K3567 | 3 | BS05001 | 0 | BS02036 | 0 |
| Y1954 | 2 | K3962 | 2 | BS05005 | 3 | BS02039 | 1 |
| K2065 | 1 | K3963 | 0 | BS04042 | 2 | BS02040 | 0 |
| K2071 | 2 | Y3962 | 0 | BS05006-1 | 2 | BS02043 | 3 |
| Y2210 | 1 | Y3958 | 1 | BS08023 | 2 | BS02044 | 3 |
| Y2213 | 1 | Y3960 | 1 | BS08024 | 2 | BS02045 | 2 |
| Y2217 | 3 | Y3964 | 0 | BS08025 | 0 | BS02047 | 2 |
| Y2245-2 | 3 | Y3965 | 2 | BS08027-1 | 2 | BS02050 | 2 |
| Y4017 | 0 | Y4210 | 2 | BS08027-2 | 0 | BS02051-1 | 2 |
| Y4020 | 2 | Y4221 | 0 | BS08028 | 0 | BS02052 | 2 |
| Y4044 | 2 | K5006 | 2 | BS08029 | 1 | BS02053 | 0 |
| Y4052 | 0 | K5011 | 2 | BS08031 | 0 | BS02055 | 1 |
| Y4055 | 2 | K5012 | 0 | BS08032 | 1 | BS02057 | 1 |
| Y4056 | 2 | K5014 | 2 | BS08034 | 2 | BS02058 | 2 |
| Y4058 | 2 | K5016-2 | 2 | BS08035 | 3 | BS02059-1 | 2 |
| Y4066 | 2 | K5017 | 1 | BS08038 | 0 | BS02059-2 | 2 |
| Y4068 | 2 | K5018 | 2 | BS08039 | 1 | BS02060-1 | 2 |
| Y4069 | 2 | K5020 | 2 | BS08041 | 0 | BS02060-2 | 1 |
| Y4071 | 0 | K5021 | 2 | BS08042 | 2 | BS02062-2 | 2 |
| Y4072 | 2 | K5024 | 2 | BS08043 | 2 | BS02063-1 | 2 |
| Y4073 | 2 | K5027 | 2 | BS08047 | 2 | BS02063-2 | 2 |
| K4086 | 2 | K5028 | 2 | BS08049 | 2 | BS02065-1 | 2 |
| K4087 | 2 | K5030 | 2 | BS08050 | 1 | BS02065-2 | 2 |
| K4089 | 0 | K5034 | 2 | BS08052 | 1 | BS02066 | 3 |
| K4170 | 0 | K5035 | 2 | BS08053 | 0 | BS02069 | 2 |
| K4177 | 0 | K5036 | 0 | BS08054 | 1 | BS02071 | 2 |
| Y4075 | 2 | K5037 | 2 | Y3908 | 2 | BS02072 | 3 |
| Y4077 | 2 | K5040 | 2 | Y3845 | 2 | BS02075-3 | 2 |
| Y4106-1 | 2 | K5042 | 2 | Y3839 | 2 | BS05006-2 | 2 |
| Y4121-2 | 2 | K5051 | 0 | Y3903 | 2 | BS05007 | 2 |
| Y4129 | 0 | K5052 | 2 | Y3827 | 2 | BS05010 | 2 |
| Y4140 | 2 | K5059 | 2 | Y3865 | 2 | BS05013-1 | 1 |
| Y4148 | 2 | K5064 | 0 | Y3857 | 2 | BS05015 | 1 |
| Y4158 | 2 | K5067-1 | 2 | Y3866 | 2 | BS05016 | 0 |
| Y4166 | 2 | K5073 | 2 | Y3817 | 2 | BS05017 | 1 |
| Y4168 | 2 | K5075 | 2 | K3863-2 | 3 | BS05019-1 | 2 |
| Y4169 | 2 | K5076 | 2 | Y3856 | 2 | BS05019-2 | 2 |
| K4181 | 0 | K5077 | 0 | Y3907 | 2 | BS05022 | 2 |
| K4185 | 3 | K5102 | 0 | K3862 | 2 | BS05023-1 | 0 |
| Y4186 | 2 | K5109 | 1 | Y3904 | 2 | BS05023-2 | 0 |
| Y4194 | 2 | K5113 | 0 | K3861-1 | 3 | BS05024 | 1 |
| Y4199 | 2 | K5114 | 1 | Y3901 | 2 | BS05029 | 2 |
| K4265 | 0 | K5115 | 3 | Y3931 | 2 | BS05030-1 | 2 |
| K4266 | 0 | K5116 | 3 | Y3909 | 2 | BS05030-2 | 2 |
| K0205 | 2 | K0712 | 1 | Y3932 | 2 | BS05033-1 | 2 |
| K0206 | 3 | K0713 | 1 | Y3938 | 2 | BS05033-2 | 2 |
| K0209 | 2 | K0715 | 1 | Y3950 | 0 | BS05034 | 2 |
| K0210 | 1 | K0726 | 1 | Y3951 | 2 | BS05040 | 3 |
| K0212 | 0 | K0729 | 1 | Y3953 | 2 | BS06005 | 2 |
| K0214 | 3 | K0730 | 1 | Y3955 | 2 | BS05039 | 3 |
| K0215 | 0 | K0735 | 1 | Y3957 | 1 | BS06020 | 2 |
| K0216 | 0 | K0738 | 1 | K4062 | 1 | BS06024 | 1 |
| K0217 | 3 | K0744 | 0 | K4063 | 3 | BS05036 | 3 |
| K0218 | 1 | K0753 | 0 | K4064-2 | 0 | BS05038 | 3 |
| K0219 | 0 | K0801 | 0 | K4069 | 0 | BS06003 | 0 |
| K0220 | 3 | K0805 | 0 | K4072 | 2 | BS05041 | 1 |
| K0303 | 3 | K0806 | 0 | K4073 | 0 | BS06002 | 2 |
| K0304 | 1 | K0807 | 0 | K4074 | 0 | BS06006 | 1 |
| K0305 | 1 | K0905 | 0 | K4075-2 | 3 | BS06013 | 0 |
| K0308 | 0 | K0906 | 1 | K4076-1 | 0 | BS06017 | 2 |
| K0402 | 1 | K0917 | 1 | K4076-2 | 3 | BS06010 | 1 |
| K0405 | 2 | K0918 | 1 | K4085 | 0 | BS06018 | 1 |
| K0406 | 3 | K0919 | 1 | K5118 | 2 | BS06012 | 0 |
| K0410 | 0 | K0920 | 1 | K5121 | 1 | BS06019 | 0 |
| K0412 | 2 | K0921 | 1 | K5206 | 2 | BS09011 | 2 |
| K0415 | 1 | K0922 | 1 | K5207-1 | 1 | BS09012 | 2 |
| K0503 | 3 | K0924 | 1 | K5207-2 | 1 | BS09013-1 | 2 |
| K0505 | 3 | K0928 | 1 | K5209 | 1 | BS09013-2 | 1 |
| K0506 | 3 | K0935 | 1 | K5215 | 0 | BS09016 | 0 |
| K0507 | 3 | K0936 | 2 | K5216 | 2 | BS09025 | 0 |
| K0508 | 3 | K0942 | 1 | K5217 | 2 | BS09028 | 1 |
| K0510 | 3 | K0949 | 1 | K5218 | 2 | BS10004 | 0 |
| K0513 | 3 | K0952 | 2 | K5219 | 0 | BS10013 | 1 |
| K0514-1 | 0 | K0955 | 3 | K5220 | 0 | BS10018 | 0 |
| K0515 | 3 | K1706 | 1 | K5221 | 2 | BS10019 | 0 |
| K0516 | 3 | K1707 | 2 | K5222 | 2 | F01 | 2 |
| K0514-2 | 0 | K0957 | 1 | K5224 | 2 | F02 | 2 |
| K0518 | 3 | K1713 | 2 | K5225 | 0 | F9505 | 2 |
| K0520 | 3 | K1715 | 3 | K5226 | 2 | F9601 | 2 |
| K0705 | 3 | K1716 | 1 | K5228 | 2 | F9602 | 2 |
| BTHP-14 | 3 | BS01048 | 2 | K5229 | 2 | BF10602-1 | 2 |
| BTHP-15 | 2 | BS01053 | 2 | K5232 | 2 | BC | 3 |
| BTHP-16 | 2 | BS01055 | 2 | K5231 | 1 | BS | 0 |
| BTHP-18 | 2 | BS01057 | 2 | K5230 | 2 | BTT | 3 |
| BTHP-19 | 1 | BS01059 | 1 | K5233 | 1 | BTa | 3 |
| BTHP-20 | 1 | BS01062 | 2 | K5234 | 2 | BTi-1 | 2 |
| MSC-1 | 0 | BS01063 | 1 | K5235 | 2 | BTi-C | 3 |
| W312 | 2 | BS01065 | 2 | K0101 | 1 | BTi-T | 3 |
| BS01002 | 2 | BS01067 | 2 | K0102 | 0 | B4039 | 1 |
| BS01003 | 2 | BS01068 | 0 | K0103 | 3 | B4041 | 1 |
| BS01004-1 | 2 | BS01070-1 | 0 | K0104 | 0 | B4050 | 1 |
| BS01008 | 2 | BS01070-2 | 1 | K0105 | 3 | B4055 | 3 |
| BS01011 | 2 | BS01071 | 0 | K0108 | 1 | Bt25601 | 3 |
| BS01012 | 2 | BS01073 | 0 | K0109 | 0 | Bt25602 | 3 |
| BS01016 | 2 | BS01074 | 0 | K0112 | 0 | Bt25603 | 3 |
| BS01017 | 2 | BS01076 | 0 | K0113 | 0 | Bt26401 | 3 |
| BS01018 | 2 | BS01078 | 0 | K0201 | 0 | K2074 | 3 |
| BS01019 | 1 | BS01078-1 | 0 | K0202 | 2 | Y2126 | 1 |
| BS01023 | 2 | BS01080 | 3 | K1717 | 3 | Y4171 | 0 |
| BS01024 | 2 | BS0108] | 2 | K1719 | 3 | K5406 | 0 |
| BS01026 | 0 | BS01083 | 1 | K1720 | 3 | BS02018 | 0 |
| BS01028-2 | 1 | BS01087 | 1 | Y2038 | 3 | BS02019 | 1 |
| BS01030 | 1 | BS01088 | 3 | K5402 | 0 | BS02021 | 2 |
| BS01032 | 1 | BS02004 | 2 | K5518 | 3 | BS03015 | 0 |
| BS01034 | 3 | BS02005 | 2 | K5701 | 0 | BS03016-1 | 0 |
| BS01035 | 1 | BS02006 | 2 | K5714 | 0 | BS03016-2 | 0 |
| BS01036 | 0 | BS02007 | 1 | K5824 | 0 | BS03020 | 0 |
| BS01038 | 1 | BS02009 | 3 | K5827 | 0 | BS08058 | 1 |
| BS01039 | 1 | BS02010 | 0 | K5828 | 0 | BS08060 | 2 |
| BS01040 | 1 | BS02011 | 1 | K5908 | 0 | BS08061 | 2 |
| BS01041 | 3 | BS02014 | 1 | K6002 | 0 | BS08063 | 2 |
| BS01043 | 0 | BS02016 | 1 | K6003 | 0 | BS08066 | 1 |
| BS01044 | 1 | BS02017 | 1 | K6122 | 0 | BS08067-1 | 2 |
| BS01045 | 0 | BS02082 | 2 | BS02093 | 0 | BS08067-2 | 2 |
| BS01046 | 2 | BS02083-1 | 2 | BS02098-2 | 3 | BS08068 | 0 |
| BS01047 | 2 | BS02083-2 | 2 | BS020100 | 0 | BS08093 | 1 |
| BS02077 | 2 | BS02085 | 0 | BS02105 | 0 | BS08097 | 3 |
| BS02078-1 | 2 | BS02091 | 1 | BS02106 | 0 | BS09005 | 2 |
| BS02078-2 | 2 | BS02092-1 | 0 | BS02108 | 0 | BS09009 | 2 |
| BS02080 | 2 | BS02092-3 | 1 | BS02116 | 0 | BS09010-1 | 2 |
| BS02081 | 3 | BS02118 | 0 | BS02119 | 0 | BS09010-2 | 1 |
| BS02120 | 1 | BS03003 | 2 | BS03006-2 | 2 | BS06027 | 3 |
| BS02123 | 2 | BS03004-1 | 2 | BS03008-1 | 0 | BS06029 | 1 |
| BS03001-1 | 2 | BS03004-2 | 2 | BS03008-2 | 1 | BS06031 | 2 |
| BS03001-2 | 2 | BS03005 | 2 | BS03012 | 1 | BS06033 | 2 |
| BS03002 | 2 | BS03006-1 | 2 | BS03013 | 0 | BS06036 | 1 |
| BS06039 | 0 | BS07002 | 0 | BS07016 | 1 | BS08023 | 2 |
| BS06040 | 2 | BS07005 | 0 | BS07020-1 | 2 | BS07024 | 1 |
| BS06045 | 3 | BS07008 | 3 | BS07020-2 | 2 | BS08002 | 0 |
| BS06049 | 3 | BS07010 | 2 | BS07020-3 | 2 | BS08003 | 2 |
| BS07001 | 1 | BS07011 | 2 | BS07022 | 3 | BS08005 | 1 |
| BS08008 | 2 | BS08010 | 1 | BS08012 | 1 | BS08014 | 3 |
| BS08009 | 2 | BS08011 | 2 | BS08013 | 2 | BS08017-2 | 3 |
| BS08018 | 2 | BS08019 | 2 | BS08021 | 3 | | |

Note:
Through eye-measuring the settlement condition for the suspended particles in the kaolin suspension, the flocculating capabilities can be categorized as four grades: 0, 1, 2 and 3. “0” represents no obvious facilitation (acceleration) for the settlement of the suspended particles, while “3” represents the increased settling rate for the particles and having the best flocculating effect.

### Example 2

### Selected Strains Subjected to High Temperature Treatment

The purpose of this experiment is to evaluate the influence on the flocculating activities of the bacterial cell precipitates, after high temperature treatments, toward the kaolin suspensions and the textile dyeing wastewater. This is necessary because spray drying, carried out under high temperature, may be used to recover biological flocculants containing the cell precipitates. Bacterial strains with flocculating effect of “3” were selected to examine the influence of high temperature on the cell precipitates' flocculating capacity.

More specifically, first, from the preliminary screening, 50 microbial strains with the potential of producing biological flocculants, i.e. flocculating effect of “3” and not under biohazard regulation restrictions, were selected. Microbes having flocculating activities toward both the kaolin suspensions and the wastewater from the textile dyeing industries were chosen. These microbes were incubated with 100 ml Nutrient Broth (Merck) in 500 ml round flasks, under 30° C., shaking in 200 rpm, for 48 hours. After the 48-hour incubation, the bacterial solution underwent high temperature treatment of 122° C. and 1.5 atm., followed by centrifugation under 4° C., 10,000 rpm for 10 minutes to collect the bacterial cell precipitate. The collected bacterial cell precipitate was diluted by sterile water to a volume of 10 ml (this bacterial solution is 10 times the concentration of the original starting bacterial solution of 100 ml).

Then, evaluation of the flocculating activity of the concentrated bacterial solution with respect to kaolin suspension and textile dyeing wastewater was carried out. Flocculation toward the kaolin suspension was examined by thoroughly mixing 5 ml of the concentrated bacterial solution, 45 ml of tap water, 0.5 ml of 3% CaCl₂.2H₂O solution and 20 ml of 1.25% kaolin suspension. The mixture was then kept still for 30 minutes. Then, changes in the suspended particles in the mixture solution and volume of the settled sludge were observed.

The evaluation of the flocculating activity of the concentrated bacterial solution toward the wastewater from the textile dyeing industry was also examined. This was performed by thoroughly mixing 5 ml of the concentrated bacterial solution with 45 ml of the textile dyeing wastewater (Li-Pong, Kai-Ling) and 1 ml of 5% poly aluminum chloride (PACl) solution. Changes in the suspended particles in the mixture solution and volume of the settled sludge volume were observed after keeping the mixture still for 30 minutes.

Through eye-measurement, the flocculating capabilities can be categorized into six grades: 0, 1, 2, 3, 4 and 5. “0” represents no obvious facilitation (acceleration) for the settlement of the suspended particles, while “5” represents the increased settling rate for the particles and having the best flocculating effects. If the floc that was formed after the application of the bacterial cell precipitate was buoyant, the bacterial cell precipitate solution was denoted as “S”, see Table 2 below. Five bacterial strains K0214, K0220, K3463, K5518 and Y2105-1 with the best flocculating capabilities (considering the effect in the textile dyeing wastewater given that results were almost the same in kaolin suspensions) and high recovery rate were selected as possible bacterial strains for producing the biological flocculants.

TABLE 2
Comparison of the flocculating activities of bacterial solutions containing
strains, with flocculating capabilities, treated by high temperature treatment

| | 121° C., 1.5 atm | | | | 121° C., 1.5 atm | | |
|---|---|---|---|---|---|---|---|
| Treatment | 20 min | | 160° C., 1 atm | Treatment | 20 min | | 160° C., 1 atm |
| Testing | | Textile dyeing | 24 hours | Testing | | Textile dyeing | 24 hours |
| solution | Kaolin | wastewater | Kaolin | solution | Kaolin | wastewater | Kaolin |
| BF11 | — | — | 2 | K1715 | 3 | 1S | 3.5 |
| BTHP-6 | 3 | 0 | 2 | K1717 | 3 | 2S | 2 |
| BTHP-12 | 3 | 2 | 2 | K1719 | 3 | 2S | 2 |
| BTHP-13 | 3 | 1 | 3 | K1720 | 3 | 1 | 2 |
| K0103 | 3 | 2S | 4 | K3463 | 3 | 3S | 5 |
| K0105 | 3 | 2S | 4 | K3567 | 3 | 1 | 1 |
| K0206 | 3 | 2 | 3 | K3861-1 | 3 | 1 | 4 |
| K0214 | 3 | 3 | 3 | K3863-2 | 3 | 1 | 5 |
| K0217 | 3 | 1 | 3 | K4063 | 3 | 1 | 4 |
| K0220 | 3 | 3 | 2 | K4075-2 | 3 | 1S | 4 |
| K0303 | 3 | 2S | 5 | K4076-2 | 3 | 1 | 2 |
| K0406 | 3 | 2S | 5 | K4185 | 3 | 2 | 1 |
| K0503 | 3 | 1S | 3 | K5115 | 3 | 2 | 1 |
| K0505 | 3 | 1 | 4 | K5116 | 3 | 1 | 2 |
| K0506 | 3 | 2 | 1 | K5518 | 3 | 3S | 1 |
| K0507 | 3 | 1 | 3 | Y1941 | 3 | 2S | 4 |
| K0508 | 3 | 2 | | Y2038 | 3 | 1S | 1.5 |
| K0510 | 3 | 0 | 2 | Y2105-1 | 3 | 3 | 5 |
| K0513 | 3 | 1 | 1 | Y2217 | 3 | 1S | 2 |
| K0515 | 3 | 1 | 2 | Y2245-2 | 3 | 1S | 4 |
| K0516 | 3 | 1 | 1.5 | Y3403-1 | 3 | 2S | 4 |
| K0518 | 3 | 1 | 0.5 | Y3404 | 3 | 0S | 1 |
| K0520 | 3 | 0 | 3 | Y3406 | 3 | 2S | 1 |
| K0667 | 3 | 1 | 2 | Y4148 | 3 | 2S | — |
| K0705 | 3 | 1 | 2 | Polymer | 2 | 3 | — |
| K0955 | 3 | 1S | 4 | PACL | t | 2 | — |
| | | | | Blank | 0 | 1 | 0 |
| Note: | | | | | | | |
| Through eye-measuring the settlement condition of the suspended particles in the kaolin suspension, the flocculating capabilities can be categorized into six grades: 0, 1, 2, 3, 4 and 5. “0” represents no obvious facilitation (acceleration) for the settlement of the suspended particles, while “5” represents the increased settling rate for the particles and having the best flocculating effects. If the floc that was formed after the application of the | | | | | | | |
| # bacterial cell precipitate was buoyant, the bacterial strain was denoted as “S”. | | | | | | | |

### Example 3

### Determination of Suitable Culture Time

Since different culture times have great influences on the flocculating capabilities of the bacterial solutions, this example sought to determine the most suitable culture time using one specific bacterial strain, strain Y2105-1. Through the observation of the flocculating activity of the bacterial cell precipitate dilution toward the wastewater from the textile dyeing industries, the influences of the culture media, with varying culture time, upon the flocculating activity of the bacterial precipitate dilution were evaluated.

The culturing method of the seed bacteria is described as follows. Bacterial strain Y2105-1 was incubated with 100 ml Nutrient Broth (Merck) in 500 ml round flasks, under 30° C., shaking in 200 rpm, with the culture time of 15 hours. This seed bacterial solution, 5 ml, was inoculated onto the 9 production culture media (as listed in Table 3, each of 100 ml) and then incubated under 30° C., shaking in 200 rpm, for 48 hours, 72 hours and 96 hours respectively. After the bacterial solution was treated under high temperature and high pressure, it was centrifuged under 4° C., 10,000 rpm for 10 minutes to collect the bacterial cell precipitate. The collected bacterial cell precipitate was diluted by sterile water to a volume of 100 ml. The flocculating activity of the bacterial cell precipitate solution toward the textile dyeing wastewater was evaluated as described below.

The five control groups used in this experiment are listed as follows:
1. Original wastewater; absorbance OD₅₅₀ was 0.29; no sludge was produced after settling for 40 minutes
2. Original wastewater with pH adjusted to 6.5˜7.0 by 3 N sulfuric acid solution; absorbance OD₅₅₀ was 0.25; no sludge was produced after settling for 40 minutes
3. Original wastewater with pH adjusted to 6.5˜7.0 by 3 N sulfuric acid solution and 5 ml of 0.05% chemical organic flocculant EA-630 (Jiu-He International) was added; absorbance OD₅₅₀ was 0.25; no sludge was produced after settling for 40 minutes
4. Original wastewater with pH adjusted to 6.5˜7.0 by 5% aluminum chloride solution; absorbance OD₅₅₀ of supernatant was 0.2 after settling for 40 minutes; the settled sludge volume was 20 ml
5. Original wastewater with pH adjusted to 6.5˜7.0 by 5% aluminum chloride solution and 5 ml of 0.05% chemical organic flocculant EA-630 (Jiu-He International) was added; absorbance OD₅₅₀ of supernatant was 0.12 after settling for 40 minutes; the settled sludge volume was 12 ml.

Different culture media, cultivating the Y2105-1 strain for different culture times, were established to determine the best culture time range for the different culture media for achieving the best flocculating activity. The major differences between various culture media were the variety of nitrogen sources and their concentrations. The detailed compositions of the culture media are listed in Table 3 below. 5 ml, and 2 ml for some culture, of the bacterial cell precipitate solution was mixed with 45 ml of the textile dyeing wastewater (Ho-Yo) and 0.25 ml of 5% poly aluminum chloride solution which was added to adjust the pH of the wastewater to 6.5˜7.0 for better performance by the bacteria. The settled sludge volume (SSV, unit: ml) and the absorbance (optical density) of the supernatant (OD₅₅₀, wavelength 550 nm) were observed after keeping the mixture still for 30 minutes.

TABLE 3
The composition of the production culture medium in one liter

| | | Main carbon | |
|---|---|---|---|
| Number | Main nitrogen source | source | Others |
| 1 | Soybean meal 6 g | Glucose 15 g | Molasses 2 g |
| 2 | Soybean meal 15 g | Glucose 15 g | Molasses 2 g |
| 3 | Fish meal 6 g | Glucose 15 g | Molasses 2 g |
| 4 | Fish meal 15 g | Glucose 15 g | Molasses 2 g |
| 5 | Skin milk 20 g | Glucose 15 g | Molasses 2 g |
| 6 | Skin milk 30 g | Glucose 15 g | Molasses 2 g |
| 7 | Soybean Protein MP-90 24 g | Glucose 15 g | Molasses 2 g |
| 8 | Soybean Protein Supro-620 30 g | Glucose 15 g | Molasses 2 g |
| 9 | NB 8 g | Glucose 15 g | Molasses 2 g |

The experimental results are listed in Table 4. With the shaking fermentation time of 96 hours, the bacterial solutions from all culture media had good flocculating effects toward the textile dyeing wastewater. The settled sludge volumes (SSV) were all within 12 ml˜20 ml, and the absorbance OD₅₅₀ of the supernatants being about 0.2. The results were not very different from that of the controls with the chemical organic flocculant.

TABLE 4
The flocculating efficiency of the bacterial precipitate dilution for the strain
Y2105-1 under various culture conditions

| | Carbon source | Culture 48 hours | | Culture 72 hours | | Culture 96 hours | | |
|---|---|---|---|---|---|---|---|---|
| Number | in culture medium | SSV (mL) | OD₅₅₀ | SSV (mL) | OD₅₅₀ | SSV (mL) | OD₅₅₀ | Note |
| 1 | Soybean meal 6 g/L | 15 | 0.31 | 15 | 0.32 | 16.5 | 0.21 | |
| 2 | Soybean meal 15 g/L | 15.5 | 0.4 | 16 | 0.42 | 20 | 0.24 | |
| 3 | Fish meal 6 g/L | 14 | 0.24 | 13.5 | 0.31 | 14 | 0.23 | |
| 4 | Fish meal 15 g/L | 17 | 0.29 | 15 | 0.33 | 13 | 0.27 | |
| 5 | Skin milk 20 g/L | 20 | 0.24 | 20.5 | 0.32 | 29 | 0.2 | |
| 6 | Skin milk 30 g/L | 20 | 0.33 | 21 | 0.4 | 20 | 0.31 | |
| 7 | Soybean Protein MP-90 24 g/L | 23.5 | 0.32 | 21 | 0.28 | 21 | 0.29 | * |
| | | | | | | 20 | 0.29 | # |
| 8 | Soybean Protein Supro-620 | 36 | 0.35 | 23 | 0.31 | 19 | 0.43 | * |
| | 30 g/L | | | | | 18 | 0.31 | # |
| 9 | Nutrient Broth | 17 | 0.23 | | | | | |

Note:
“*” denotes the usage amount of 5 ml,
“#” denotes the usage amount of 2 ml, and SSV is the settled sludge volume.

### Example 4

### Determination of Suitable Composition of the Culture Medium

The purpose of this study is to compare, changes in the flocculating activities of the bacterial precipitate dilution and the bacterial solution (containing the nutrient broth) for strain Y2105-1 and strain K5518 cultivated under the most suitable culture time, so that the influence of the culture medium upon the flocculating activities of each type of solution can be evaluated.

Steps were taken to prepare various culture media. The seed culture medium was 100 ml Nutrient Broth (Merck), with the culture conditions being under 30° C., shaking in 200 rpm, culturing for 15 hours. The seed bacterial solution, 5 ml, was inoculated onto 13 different production culture media (as listed in Table 5, each in 100 ml) and then incubated under 30° C., shaking in 200 rpm, for 96 hours. Each bacterial solution was centrifuged under 4° C., 10,000 rpm for 10 minutes to collect the bacterial cell precipitate. The collected bacterial cell precipitate was diluted by tap water to 5 times of the bacterial cell precipitate volume.

TABLE 5
The composition formulation of each production culture medium in
one liter

| | | Main carbon | |
|---|---|---|---|
| Number | Main nitrogen source | source | Others |
| 1 | Soybean meal 6 g | Glucose 15 g | Molasses 2 g |
| 2 | Soybean meal 15 g | Glucose 15 g | Molasses 2 g |
| 3 | Fish meal 6 g | Glucose 15 g | Molasses 2 g |
| 4 | Fish meal 15 g | Glucose 15 g | Molasses 2 g |
| 5 | Skin milk 6 g | Glucose 15 g | Molasses 2 g |
| 6 | Skin milk 15 g | Glucose 15 g | Molasses 2 g |
| 7 | Skin milk 30 g | Glucose 15 g | Molasses 2 g |
| 8 | Soybean Protein Supro-620 6 g | Glucose 15 g | Molasses 2 g |
| 9 | Soybean Protein Supro-620 15 g | Glucose 15 g | Molasses 2 g |
| 10 | Soybean Protein Supro-620 30 g | Glucose 15 g | Molasses 2 g |
| 11 | Soybean Protein MP-90 6 g | Glucose 15 g | Molasses 2 g |
| 12 | Soybean Protein MP-90 15 g | Glucose 15 g | Molasses 2 g |
| 13 | Soybean Protein MP-90 24 g | Glucose 15 g | Molasses 2 g |

There were five control groups in this experiment and the analysis results of the supernatant and the settled sludge are described as follows:
1. Original wastewater; absorbance OD₅₅₀ was 0.26; no sludge was produced after settling for 40 minutes.
2. Original wastewater with the pH adjusted to 6.5˜7.0 by 3 N sulfuric acid solution; absorbance OD₅₅₀ was 0.23; no sludge was produced after settling for 40 minutes.
3. Original wastewater with the pH adjusted to 6.5˜7.0 by 3 N sulfuric acid solution and 5 ml of 0.05% chemical organic flocculant EA-630 (Jiu-He International) was added; absorbance OD₅₅₀ was 0.18; no observable sludge was produced after settling for 40 minutes.
4. Original wastewater with the pH adjusted to 6.5˜7.0 by 5% aluminum chloride solution; absorbance OD₅₅₀ was 0.03 after settling for 40 minutes; the settled sludge volume was 22 ml.
5. Original wastewater with the pH adjusted to 6.5˜7.0 by 5% aluminum chloride solution and 5 ml of 0.05% chemical organic flocculant EA-630 (Jiu-He International) was added; absorbance OD₅₅₀ was 0.03 after settling for 40 minutes; the settled sludge volume was 13 ml.

The flocculating activity was then initiated to examine the effect. 1 ml and 5 ml of the bacterial precipitate dilutions were each mixed with 45 ml of the textile dyeing wastewater (Ho-Yo) and 0.25 ml of 5% poly aluminum chloride solution to adjust the pH of the wastewater to 6.5˜7.0. The settled sludge volume and the absorbance (optical density, OD₅₅₀) of the supernatant were observed after keeping the mixture still for 30 minutes.

The experimental results are listed in Table 6 and Table 7. The strain Y2105-1 had better flocculating effects than the strain K551.8, because the absorbance of the supernatant and the settled sludge volume of the wastewater that was treated by the Y2105-1 bacterial precipitate dilution were lower than those of the wastewater that was treated by the K5188 bacterial precipitate dilution. For the Y2105-1 strain cultivated in media with the carbon source Supro-620 and MP-90, 5 ml of its bacterial precipitate dilution had the flocculating activity toward the wastewater comparable to that of the control with the addition of the chemical organic flocculant, with the absorbance OD₅₅₀ less than 0.1.

TABLE 6
The flocculating efficiency of the bacterial precipitate dilution for the strain
Y2105-1 with various culture medium compositions and 96-hour culture time

| | | Bacterial cell | Bacterial precipitate | | Bacterial precipitate | |
|---|---|---|---|---|---|---|
| | Carbon source | production | dilution 5 ml | | dilution 1 ml | |
| Number | in culture medium | (g/L) | SSV (mL) | OD₅₅₀ | SSV (mL) | OD₅₅₀ |
| 1 | fish meal 6 g/L | 2.52 | 13 | 0.78 | 12 | 0.29 |
| 2 | fish meal 15 g/L | 3.89 | 15 | 1.38 | 12 | 0.39 |
| 3 | soybean meal 6 g/L | 2.74 | 20 | 0.11 | 13.5 | 0.14 |
| 4 | soybean meal 15 g/L | 5.25 | 24 | 0.11 | 14 | 0.15 |
| 5 | skin milk 6 g/L | 2.8 | 16 | 0.11 | 13 | 0.11 |
| 6 | skin milk 15 g/L | 1.97 | 23 | 0.48 | 21 | 0.18 |
| 7 | skin milk 30 g/L | 10.01 | 14 | 0.5 | 13.5 | 0.21 |
| 8 | Super-620 6 g/L | 4.93 | 29 | 0.05 | 13.5 | 0.11 |
| 9 | Super-620 15 g/L | 10.82 | 32 | 0.06 | 14.5 | 0.11 |
| 10 | Super-620 30 g/L | 17.5 | 37 | 0.06 | 15 | 0.1 |
| 11 | MP-90 6 g/L | 4.65 | 34 | 0.04 | 13 | 0.1 |
| 12 | MP-90 15 g/L | 10.16 | 39 | 0.04 | 16 | 0.12 |
| 13 | MP-90 24 g/L | 16.47 | 34 | 0.08 | 15 | 0.13 |

TABLE 7
The flocculating efficiency of the bacterial precipitate dilution
for the strain K5518 with various culture medium
compositions and 96-hour culture time

| | | | Bacterial | | Bacterial | |
|---|---|---|---|---|---|---|
| | | Bacterial | precipitate | | precipitate | |
| | | cell | dilution 5 ml | | dilution 1 ml | |
| | Carbon source | production | SSV | | SSV | |
| Number | in culture medium | (g/L) | (mL) | OD₅₅₀ | (mL) | OD₅₅₀ |
| 1 | Fish 6 | 2.63 | 34 | 2.55 | 13 | 0.37 |
| 2 | Fish 15 | 3.89 | 42 | 2.2 | 14.5 | 0.2 |
| 3 | Soybean meal 6 | 2.74 | 21 | 1.78 | 15 | 0.34 |
| 4 | Soybean meal 15 | 5.25 | 24 | 0.79 | 34 | 0.13 |
| 5 | Skin milk 6 | 1.68 | 41.5 | 2.46 | 37 | 0.19 |
| 6 | Skin milk 15 | 3.63 | 12.5 | 2.27 | 37 | 0.29 |
| 7 | Skin milk 30 | 4.21 | 35 | 0.2 | 16 | 0.16 |
| 8 | Super 6 | 4.34 | 42 | 0.72 | 31 | 0.1 |
| 9 | Super 15 | 2.35 | 13 | 3.02 | 13 | 0.59 |
| 10 | Super 30 | 3.26 | 14 | 3.1 | 13 | 1.19 |
| 11 | MP-90 6 | 5.3 | 4. | 0.8 | 17 | 0.12 |
| 12 | MP-90 15 | 3.1 | 12 | 2.95 | 12 | 0.82 |
| 13 | MP-90 24 | 3.42 | 13 | 3.14 | 12 | 1.01 |

The above experimental results show that the bacterial precipitate dilutions from the bacteria cultured with Supro-620 and MP-90 of soybean protein had better flocculating activities toward the wastewater.

### Example 5

### Verification of Best Culture Medium for Industrial Production

The purpose of this experiment is to verify the best culture medium for industrial production for the strains Y2105-1, K5518, K3463, K0214 and K0220 cultured under the most suitable culture time, and to evaluate the practicability of producing biological flocculants by using the lactic fermentation wastewater.

Flocculating activity testing solutions were prepared. The seed culture medium was 100 ml Nutrient Broth (Merck), with the culture conditions being under 30° C., shaking in 200 rpm, under the culture time of 15 hours. 5 ml of the seed bacterial solution was inoculated onto each of the 6 production culture media (as listed in Table 8, each medium in 100 ml) and 100 ml of the lactic fermentation wastewater and then incubated under 30° C., shaking in 200 rpm, for 96 hours. Each bacterial solution (90 ml) was centrifuged under 4° C., 10,000 rpm for 10 minutes to collect the bacterial cell precipitate. The collected bacterial cell precipitate was diluted by tap water to 5 times of the bacterial cell precipitate volume.

TABLE 8
The composition formulation of the production culture medium in
one liter

| | | Main carbon | |
|---|---|---|---|
| Number | Main nitrogen source | source | Others |
| 1 | Soybean Protein Supro-620 15 g | Glucose 15 g | Molasses 2 g |
| 2 | Soybean Protein Supro-620 15 g | Glucose 15 g | Molasses 5 g |
| 3 | Soybean Protein Supro-620 15 g | Corn Starch | Molasses 5 g |
| | | 15 g | |
| 4 | Soybean Protein MP-90 15 g | Glucose 15 g | Molasses 2 g |
| 5 | Soybean Protein MP-90 15 g | Glucose 15 g | Molasses 5 g |
| 6 | Soybean Protein MP-90 15 g | Corn Starch | Molasses 5 g |
| | | 15 g | |
| 7 | Lactic fermentation waste 100% | | |

There were five controls groups for this study and are described as follows:
1. Original wastewater with pH at 8.53; absorbance OD₅₅₀ was 0.17; no sludge was produced after settling for 40 minutes.
2. Original wastewater with the pH adjusted to 6.5˜7.0 by 3 N sulfuric acid solution; absorbance OD₅₅₀ was 0.15; no sludge was produced after settling for 40 minutes.
3. Original wastewater with the pH adjusted to 6.5˜7.0 by 3 N sulfuric acid solution and 5 ml of 0.05% chemical organic flocculant EA-630 (Jiu-He International) was added; absorbance OD₅₅₀ was 0.15; no observable sludge was produced after settling for 40 minutes.
4. Original wastewater with the pH adjusted to 6.5˜7.0 by 5% aluminum chloride solution; absorbance OD₅₅₀ was 0.04 after settling for 40 minutes; the settled sludge volume was 35 ml.
5. Original wastewater with the pH adjusted to 6.5˜7.0 by 5% aluminum chloride solution and 5 ml of 0.05% chemical organic flocculant EA-630 (Jiu-He International) was added; absorbance OD₅₅₀ was 0.06 after settling for 40 minutes; the settled sludge volume was 12 ml.

Then the evaluation of the flocculating activity was carried out. 45 ml of the textile dyeing wastewater (Ho-Yo) and 0.25 ml of 5% poly aluminum chloride solution were mixed with 5 ml and 1 ml of the bacterial solution and 1 ml of the bacterial precipitate dilution respectively. Changes in the settled sludge volume and the absorbance (OD₅₅₀) of the supernatant were observed after keeping the mixture still for 30 minutes.

The experimental results are listed in Table 9. The results show that the bacterial precipitate dilutions and the bacterial solutions obtained from all of the bacterial strains that were cultivated with the lactic fermentation waste had excellent flocculating capabilities toward the wastewater from the textile dyeing industries. However, because bacterial cell production under such cultivation was low and the source of the lactic fermentation waste is unstable, lactic fermentation wastewater is not as suitable to be used as a raw material for producing the biological flocculants. For the flocculating capabilities of the biological flocculants obtained from using the other commercialized culture media, the test results show that strains Y2105-1 and K0214 has effects comparable to that of control group 5. The following three compositions had the most pronounced effects:
1. When strain K0214 was produced (cultivated) with culture medium C (Supro-620 15 g/L, corn starch 15 g/L, molasses 5 g/L), the dosage of 1 ml bacterial solution resulted in settled-sludge volume (SSV) of 16 ml and absorbance OD₅₅₀ of 0.07.
2. When strain Y2105-1 was produced (cultivated) with culture medium A (Supro-620 15 g/L, glucose 15 g/L, molasses 2 g/L), the dosage of 1 ml bacterial solution resulted in settled sludge volume (SSV) of 16 ml and absorbance OD₅₅₀ of 0.08.

3. When strain Y2105-1 was produced (cultivated) with culture medium E (MP-90 15 g/L, glucose 15 g/L, molasses 5 g/L), the dosage of 1 ml bacterial solution resulted in settled sludge volume (SSV) of 16 ml and absorbance OD₅₅₀ of 0.08.

TABLE 9
Comparison of the flocculating efficiency between the bacterial precipitate
dilutions and the bacterial cell precipitates for various strains

| | Culture | Bacterial solution | | Bacterial solution | | Bacterial precipitate | |
|---|---|---|---|---|---|---|---|
| Strain | medium | 5 ml | | 1 ml | | dilution 1 ml | |
| number | composition | SSV (mL) | OD₅₅₀ | SSV (mL) | OD₅₅₀ | SSV (mL) | OD₅₅₀ |
| K0214 | A | 43 | 0.29 | 18 | 0.1 | 32 | 0.06 |
| | B | 42 | 0.23 | 21 | 0.09 | 27 | 0.05 |
| | C | 34 | 0.07 | 16 | 0.07 | 34 | 0.04 |
| | D | 40 | 0.29 | 18 | 0.09 | 29 | 0.06 |
| | E | 40 | 0.34 | 18 | 0.09 | 31 | 0.06 |
| | F | 31 | 0.08 | 16 | 0.08 | 19 | 0.1 |
| | G | 23 | 0.06 | 16 | 0.06 | 17 | 0.08 |
| K0220 | A | 13 | ND | 13 | ND | 24 | ND |
| | B | 18 | ND | 13 | ND | 19 | ND |
| | C | 15 | ND | 12 | ND | 15 | ND |
| | D | 40 | 0.21 | 17 | 0.09 | 20 | 0.1 |
| | E | 20 | ND | 13 | ND | 8 | ND |
| | F | 18 | ND | 11 | ND | 15 | ND |
| | G | 24 | ND | 16 | 0.07 | 12 | 0.06 |
| K3463 | A | 14 | ND | 13 | ND | 14 | ND |
| | B | 13 | ND | 13 | ND | 13 | ND |
| | C | 21 | ND | 23 | ND | 22 | ND |
| | D | 16 | ND | 13 | ND | 12 | ND |
| | E | 18 | ND | 12 | ND | 10 | ND |
| | F | 16 | ND | 12 | ND | 13 | ND |
| | G | 22 | 0.05 | 16 | 0.06 | 12 | 0.06 |
| K5518 | A | 16 | ND | 22 | 0.17 | 28 | 0.08 |
| | B | 20 | ND | 26 | 0.16 | 27 | 0.1 |
| | C | 15 | ND | 19 | 0.35 | 21 | 0.08 |
| | D | 22 | ND | 27 | 0.14 | 14 | 0.35 |
| | E | 17 | ND | 28 | 0.18 | 18 | 0.1 |
| | F | 19 | ND | 16 | 0.4 | 15 | 0.14 |
| | G | 23 | 0.06 | 16 | 0.06 | 9 | 0.06 |
| Y2105-1 | A | 32 | 0.07 | 16 | 0.08 | 33 | 0.04 |
| | B | 31 | 0.1 | 16 | 0.09 | 29 | 0.03 |
| | C | 28 | 0.24 | 12 | 0.13 | 26 | 0.04 |
| | D | 32 | 0.07 | 16 | 0.07 | 33 | 0.05 |
| | E | 40 | 0.08 | 16 | 0.08 | 14 | 0.07 |
| | F | 25 | ND | 14 | 0.16 | 11 | 0.07 |
| | G | 25 | 0.05 | 16 | 0.06 | 11 | 0.06 |

Note:
1. ND (no data) represents that the turbidity of the suspension was too high to be measured.
2. The composition of the culture medium A: Supro-620 15 g/L, glucose 15 g/L, molasses 2 g/L.
3. The composition of the culture medium B: Supro-620 15 g/L, glucose 15 g/L, molasses 5 g/L.
4. The composition of the culture medium C: Supro-620 15 g/L, corn starch 15 g/L, molasses 5 g/L.
5. The composition of the culture medium D: MP-90 15 g/L, glucose 15 g/L, molasses 2 g/L.
6. The composition of the culture medium E: MP-90 15 g/L, glucose 15 g/L, molasses 5 g/L.
7. The composition of the culture medium F: MP-90 15 g/L, corn starch 15 g/L, molasses 5 g/L.
8. The composition of the culture medium G: 100% lactic fermentation waste solution.

### Example 6

### Verification of Flocculants Producing Microbial Strains

One purpose of this study is to verify the bacterial strains that produce biological flocculants. The flocculating activities of the bacterial solutions containing strain Y2105-1 and the strain K0214 in various industrial production culture media listed in Table 10 and Table 11 with the culture time of 96 hours toward the industrial wastewater were compared. The two strains were selected because of their better flocculating capabilities and high recovery rate.

The testing solutions were prepared as follows. The seed culture medium was 100 ml Nutrient Broth (Merck), with the culture conditions being under 30° C., shaking in 200 rpm, and culturing for 15 hours. 5 ml of the seed bacteria solution was inoculated onto the production culture media. Based on culture media A,

C and E described in Example 5, the carbon source and the molasses concentration were adjusted to test the bacterial production amount and the flocculating capabilities of the bacterial solutions, thus determining the most suitable composition of the production culture media for producing the biological flocculants. The production culture media, each of 100 ml, are listed in Table 10 (for strain K0214) and in Table 11 (for strain Y2105-1). They were incubated under 30° C., shaking in 200 rpm, for 96 hours.

TABLE 10
The composition formulation of the culture medium for strain K0214

| | Soybean | Corn | |
|---|---|---|---|
| Number | Protein | Starch | Molasses |
| K1 | Supro-620 | 10 g/L | 0 g/L |
| K2 | 15 g/L | | 5 g/L |
| K3 | | | 10 g/L |
| K4 | | 15 g/L | 0 g/L |
| K5 | | | 5 g/L |
| K6 | | | 10 g/L |
| K7 | | 20 g/L | 0 g/L |
| K8 | | | 5 g/L |
| K9 | | | 10 g/L |
| K10 | Supro-620 | 10 g/L | 0 g/L |
| K11 | 25 g/L | | 5 g/L |
| K12 | | | 10 g/L |
| K13 | | 15 g/L | 0 g/L |
| K14 | | | 5 g/L |
| K15 | | | 10 g/L |
| K16 | | 20 g/L | 0 g/L |
| K17 | | | 5 g/L |
| K18 | | | 10 g/L |

TABLE 11
The composition formulation of the culture medium for strain Y2105-1

| | Soybean | Corn | |
|---|---|---|---|
| Number | Protein | Starch | Molasses |
| 1 | Supro-620 | 10 g/L | 0 g/L |
| 2 | 15 g/L | | 2 g/L |
| 3 | | | 5 g/L |
| 4 | | 15 g/L | 0 g/L |
| 5 | | | 2 g/L |
| 6 | | | 5 g/L |
| 7 | | 20 g/L | 0 g/L |
| 8 | | | 2 g/L |
| 9 | | | 5 g/L |
| 10 | Supro-620 | 10 g/L | 0 g/L |
| 11 | 25 g/L | | 2 g/L |
| 12 | | | 5 g/L |
| 13 | | 15 g/L | 0 g/L |
| 14 | | | 2 g/L |
| 15 | | | 5 g/L |
| 16 | | 20 g/L | 0 g/L |
| 17 | | | 2 g/L |
| 18 | | | 5 g/L |
| 19 | MP-90 | 10 g/L | 0 g/L |
| 20 | 15 g/L | | 5 g/L |
| 21 | | | 10 g/L |
| 22 | | 15 g/L | 0 g/L |
| 23 | | | 5 g/L |
| 24 | | | 10 g/L |
| 25 | | 20 g/L | 0 g/L |
| 26 | | | 5 g/L |
| 27 | | | 10 g/L |
| 28 | MP-90 | 10 g/L | 0 g/L |
| 29 | 25 g/L | | 5 g/L |
| 30 | | | 10 g/L |
| 31 | | 15 g/L | 0 g/L |
| 32 | | | 5 g/L |
| 33 | | | 10 g/L |
| 34 | | 20 g/L | 0 g/L |
| 35 | | | 5 g/L |
| 36 | | | 10 g/L |

There were five controls described as follows:
1. Original wastewater; absorbance OD₅₅₀ was 0.15; no sludge was produced after settling for 40 minutes.
2. Original wastewater With the pH adjusted to 6.5˜7.0 by 3 N sulfuric acid solution; absorbance OD₅₅₀ was 0.12; no sludge was produced after settling for 40 minutes.
3. Original wastewater with the pH adjusted to 6.5˜7.0 by 3 N sulfuric acid solution and 5 ml of 0.05% chemical organic flocculant EA-630 (Jiu-He International) was added; absorbance OD₅₅₀ was 0.11; the settled sludge volume was 1 ml after settling for 40 minutes.
4. Original wastewater with the pH adjusted to 6.5˜7.0 by 5% aluminum chloride solution; absorbance OD₅₅₀ was 0.03 after settling for 40 minutes; the settled sludge volume was 21 ml.
5. Original wastewater with the pH adjusted to 6.5˜7.0 by 5% aluminum chloride solution and 5 ml of 0.05% chemical organic flocculant EA-630 (Jiu-He International) was added; absorbance OD₅₅₀ was 0.03 after settling for 40 minutes; the settled sludge volume was 23 ml.

Evaluation of the flocculating activity was carried out by mixing 0.5 ml and 1 ml, respectively, of the bacterial solution with 45 ml of the textile dyeing wastewater (Ho-Yo) and 0.25 ml of 5% poly aluminum chloride solution. Changes in the settled sludge volume and the absorbance (OD₅₅₀) of the supernatant were observed after keeping the mixture still for 30 minutes.

For the fermentation solutions of strains Y2105-1 and K0214 with different production culture media, the test results of their flocculating activities are listed in Table 12 (Y2105-1) and Table-13 (K0214). Compared with the chemical treatment methods, the wastewater treated by the bacterial solutions had less settled sludge volume (SSV), but had higher absorbance OD₅₅₀ for the supernatants. For strain Y2105-1, bacteria produced from the MP-90 containing culture media had inferior treatment efficiency than those produced from the Supro-620 containing culture media. Also, with respect to strain Y2105-1, the best flocculating capability was obtained from the culture medium composition containing Supro-620 15 g/L, glucose 15 g/L and molasses 5 g/L, and the wastewater treatment resulted in settled sludge volume of 15ml and absorbance OD₅₅₀ of 0.09. With respect to strain K0214, the best flocculating capability was obtained from the culture medium composition containing Supro-620 25 g/L and corn starch 10 g/L, and the wastewater treatment resulted in settled sludge volume of 13 ml and absorbance OD₅₅₀ of 0.17. Therefore, both strains Y2105-1 and K0214 were confirmed biological flocculant producing microbes.

TABLE 12
The flocculating efficiency of the bacterial solutions for strain Y2105-1
under various culture medium compositions

| | Bacterial solution | | Bacterial solution | | | | Bacterial solution | |
|---|---|---|---|---|---|---|---|---|
| | 1 ml | | 0.5 mL | | Solid content | | 1 mL | |
| Number | SSV (mL) | OD₅₅₀ | SSV (mL) | OD₅₅₀ | (g/L) | Number | SSV (mL) | OD₅₅₀ |
| Y1 | 13.5 | 0.1 | | | 8.28 | Y19 | 11 | 0.15 |
| Y2 | 15 | 0.09 | | | | Y20 | 11 | 0.15 |
| Y3 | 15 | 0.09 | | | | Y21 | 11 | 0.18 |
| Y4 | 14 | 0.1 | | | | Y22 | 11.5 | 0.14 |
| Y5 | 15 | 0.1 | | | | Y23 | 12 | 0.15 |
| Y6 | 15 | 0.09 | | | | Y24 | 10.5 | 0.31 |
| Y7 | 11.5 | 0.14 | | | | Y25 | 11 | 0.14 |
| Y8 | 12 | 0.12 | | | | Y26 | 11.5 | 0.15 |
| Y9 | 13 | 0.14 | | | | Y27 | 10.5 | 0.32 |
| Y10 | 14 | 0.12 | | | | Y28 | 13 | 0.28 |
| Y11 | 14.5 | 0.12 | | | | Y29 | 12 | 0.15 |
| Y12 | 16 | 0.11 | 14.5 | 0.14 | | Y30 | 10 | 0.24 |
| Y13 | 11 | 0.19 | | | | Y31 | 12 | 0.22 |
| Y14 | 12 | 0.21 | | | | Y32 | 13 | 0.23 |
| Y15 | 16 | 0.12 | 14 | 0.18 | | Y33 | 10 | 0.37 |
| Y16 | 12 | 0.17 | | | | Y34 | 11.5 | 0.23 |
| Y17 | 12 | 0.2 | | | | Y35 | 12.5 | 0.18 |
| Y18 | 14 | 0.2 | | | | Y36 | 11 | 0.4 |

Note:
1. All the culture medium compositions are listed in Table 11.
2. The culture medium compositions with the higher bacterial cell production were selected to measure the weight of the solids in the fermentation solutions.

TABLE 13
The flocculating efficiency of the bacterial solutions for strain
K0214 under various culture medium compositions

| | Bacterial | | Bacterial | | |
|---|---|---|---|---|---|
| | solution | | solution | | |
| | 1 mL | | 0.5 mL | | Solid |
| | SSV | | SSV | | content |
| Number | (mL) | OD₅₅₀ | (mL) | OD₅₅₀ | (g/L) |
| K1 | 12 | 0.13 | 12.5 | 0.13 | 6.11 |
| K2 | 13 | 0.16 | | | |
| K3 | 11 | 0.17 | | | |
| K4 | 12.5 | 0.14 | 12.5 | 0.14 | 6.68 |
| K5 | 12.5 | 0.18 | | | |
| K6 | 12 | 0.18 | | | |
| K7 | 13 | 0.15 | 12.5 | 0.16 | 6.86 |
| K8 | 12 | 0.16 | | | |
| K9 | 11.5 | 0.19 | | | |
| K10 | 13 | 0.17 | 0.15 | 11.58 | |
| K11 | 13.5 | 0.17 | | | |
| K12 | 12.5 | 0.21 | | | |
| K13 | 14 | 0.14 | 14 | 0.19 | 11.46 |
| K14 | 13 | 0.19 | | | |
| K15 | 12 | 0.2 | | | |
| K16 | 13 | 0.15 | 13 | 0.15 | 11.22 |
| K17 | 13 | 0.18 | | | |
| K18 | 12 | 0.24 | | | |

Note:
1. All the culture medium compositions are listed in Table 10.
2. The culture medium compositions with the higher bacterial cell production were selected to measure the weight of the solids in the fermentation solutions.

### Example 7

### Suitable Culture Time for Strain Y2105-1 and K0214

One purpose of this study is to compare the influences on the flocculating capabilities of strains Y2105-1 and K0214 toward the industrial wastewater employing the production formulations obtained from Example 6 under the culture time of 48 hours, 72 hours, 96 hours and 120 hours, so that the most suitable culture time for strains Y2105-1 and K0214 can be determined.

Culture media were prepared. The seed culture medium was 100 ml Nutrient Broth (Merck), with the culture conditions being under 30° C., shaking in 200 rpm, with the culture time of 15 hours. The seed bacterial solution, 5 ml, was inoculated onto the production culture media (listed in Table 14, each of 100 ml), and then incubated under 30° C., shaking in 200 rpm, for 48 hours, 72 hours, 96 hours and 120 hours. Afterwards, the flocculating activities of the bacterial solutions toward the industrial wastewater were tested.

TABLE 14
The formulations of the production culture media for bacteria strains

| | | Main carbon | |
|---|---|---|---|
| Number | Main nitrogen source | source | Others |
| Y2105-1 | Soybean Protein Supro-620 15 g/L | Glucose | Molasses |
| | | 10 g/L | 5 g/L |
| K0214 | Soybean Protein Supro-620 25 g/L | Corn Starch | |
| | | 10 g/L | |
| K0214 | Soybean Protein Supro-620 25 g/L | Corn Starch | |
| | | 20 g/L | |

The controls for this study are described as follows:
1. Original wastewater with pH at 7.84; absorbance OD₅₅₀ was 0.39; the settled sludge volume was 0.1 ml after settling for 40 minutes.
2. Original wastewater with the pH adjusted to 6.64 by 3 N sulfuric acid solution; absorbance OD₅₅₀ was 0.42; the settled sludge volume was 0.1 ml after settling for 40 minutes.
3. Original wastewater with the pH adjusted to 6.64 by 3 N sulfuric acid solution and 5 ml of 0.05% chemical organic flocculent EA-630 (Jiu-He International) was added; absorbance OD₅₅₀ was 0.37; the settled sludge volume was 0.1 ml after settling for 40 minutes.
4. Original wastewater with the pH adjusted to 6.69 by 5% aluminum chloride solution; absorbance OD₅₅₀ was 0.21 after settling for 40 minutes; the settled sludge volume was 38 ml.
5. Original wastewater with the pH adjusted to 6.69 by 5% aluminum chloride solution and 5 ml of 0.05% chemical organic flocculant EA-630 (Jiu-He International) was added; absorbance OD₅₅₀ was 0.17 after settling for 40 minutes; the settled sludge volume was 38 ml.
6. Original wastewater with the pH adjusted to 6.69 by 5% aluminum chloride solution and 1 ml of 0.05% chemical organic flocculant EA-630 (Jiu-He International) was added; absorbance OD₅₅₀ was 0.33 after settling for 40 minutes; the settled sludge volume was 16 ml.

The flocculating capability of the two strains was then evaluated. 0.5 ml and 1 ml of the bacterial solution were each separately mixed with 45 ml of the textile dyeing wastewater (Ho-Yo) and 0.25 ml of 5% poly aluminum chloride solution. The settled sludge volume and the absorbance (OD₅₅₀) of the supernatant were observed after keeping the mixture still for 30 minutes.

The results of the experiment were analyzed and compared with that of the controls with the addition of the chemical organic flocculant. The extension of fermentation time from 48 hours to 120 hours had no evident influence on the flocculating activities of the bacterial solutions or the bacterial precipitate dilutions. The settled sludge volumes of all of the experimental groups were between 10 ml to 16.5 ml, while absorbance. OD₅₅₀ was about 0.3. It is concluded that the most suitable fermentation time is 72 hours when taking into account the fermentation costs, while fermentation for 96 hours requires longer occupancy time with no significant increase in flocculating effects.

TABLE 15
Comparison of the flocculating efficiency for strains Y2105-1
and K0214 under various culture time

| | | | | | Bacterial cell | | Bacterial cell | | |
|---|---|---|---|---|---|---|---|---|---|
| | Bacterial solution | | Bacterial solution | | suspension | | suspension | | |
| Culture | 1 mL | | 0.5 mL | | 1 mL | | 0.5 mL | | Solid |
| Time | SSV (mL) | OD₅₅₀ | SSV (mL) | OD₅₅₀ | SSV (mL) | OD | SSV₅₅₀ (mL) | OD | (g/L) |

Y2105-1: Supro-620 15 g/L, glucose 10 g/L, molasses 5 g/L

| | | | | | | | | | |
|---|---|---|---|---|---|---|---|---|---|
| 48 | 15 | ND | 18 | ND | 18 | 0.26 | 10 | 0.32 | 7.96 |
| 72 | 21 | 0.44 | 19 | ND | 16 | 0.23 | 11 | 0.3 | 8.43 |
| 96 | 23 | 0.4 | 26.5 | 0.32 | 15 | 0.26 | 10 | 0.32 | 8.49 |
| 120 | 24 | 0.4 | 24 | 0.35 | 15 | 0.26 | 10 | 0.3 | 8.17 |

K0214: Supro-620 25 g/L, corn starch 10 g/L

| | | | | | | | | | |
|---|---|---|---|---|---|---|---|---|---|
| 48 | 28 | 0.46 | 23 | 0.41 | 30 | 0.21 | 13.5 | 0.25 | 8.15 |
| 72 | 24 | 0.43 | 20.5 | 0.39 | 28 | 0.17 | 15 | 0.28 | 9.23 |
| 96 | 20 | 0.4 | 18 | ND | 29 | 0.16 | 15 | 0.31 | 6.77 |
| 120 | 19 | 0.47 | 19 | ND | 26.5 | 0.18 | 12 | 0.28 | 8.13 |

K0214: Supro-620 25 g/L, corn starch 20 g/L

| | | | | | | | | | |
|---|---|---|---|---|---|---|---|---|---|
| 48 | 11 | ND | 14 | ND | 30 | 0.19 | 16.5 | 0.32 | 11.78 |
| 72 | 11 | ND | 14 | ND | 28 | 0.17 | 14 | 0.28 | 8.73 |
| 96 | 11 | ND | 13 | ND | 25 | 0.14 | 15 | 0.29 | 8.73 |
| 120 | 12 | ND | 14 | ND | 22 | 0.16 | 14 | 0.26 | 10.63 |

### Example 8

### Powdered Biological Flocculants

This experiment studied the production of powdered biological flocculants from strains Y2105-1 and K0214. Commercialized organic flocculants, in general, can be categorized into two types: liquid form or powder form. Treatment plants usually have suitable equipment for the application of both types of flocculants. Because biological flocculants in powder form have longer storage life, biological flocculants products are usually developed as the powdered flocculants. The object of flocculants experiment is to compare the differences in flocculating activities of biological flocculants produced from strains Y2105-1 and K0214 that undergo spray drying to become powders.

From the results in Example 7, it is verified that the production conditions of biological flocculants for the strains K0214 and Y2105-1 are as follows:
1. The strain Y2105-1: soybean protein Supro-620 15 g/L, glucose 10 g/L and molasses 5 g/L, incubation for 96 hours.
2. The strain K0214: soybean protein Supro-620 25 g/L and corn starch 10 g/L, incubation for 96′ hours.

Test solutions were prepared. The seed culture medium was 100 ml Nutrient Broth (Merck), with the culture conditions being under 30° C., shaking in 200 rpm, with the culture time of 15 hours. The seed bacterial solution, 50 ml, was inoculated onto the production culture media (listed in Table 16, each of 1000 ml), and then incubated under 30° C., shaking in 200 rpm, for 72 or 96 hours. Afterwards, the bacterial solutions were fabricated into powders by the spray dryer (EYELA, Spray Dryer SD-1) and the flocculating activities of these two powdered biological flocculants toward the textile dyeing wastewater were tested. The inlet and outlet temperatures of the spray dryer were set to 110° C. and 90° C. respectively.

TABLE 16
The composition formulations of the production culture media for
all strains

| | | Main carbon | |
|---|---|---|---|
| Number | Main nitrogen source | source | Others |
| Y2105-1 | Soybean Protein Supro-620 15 g/L | Glucose | Molasses |
| | | 10 g/L | 5 g/L |
| K0214 | Soybean Protein Supro-620 25 g/L | Corn Starch | |
| | | 10 g/L | |

For bacterial solutions produced according to these conditions, solid content was measured. The content of the solids was 2.4% in the bacterial solution of the strain Y2105-1 and 2.9% in the bacterial solution of the strain K0214. After spray drying, the content of the solids was 86.6% in the bacterial solution of the strain Y2105-1 and 89.6% in the bacterial solution of the strain K0214.

The analysis results of the water qualities for the double controls in this experiment are as follows:
1. Original wastewater with pH at 7.84; absorbance OD₅₅₀ was 0.29, 0.26 respectively; no sludge was produced after settling for 40 minutes.
2. Original wastewater with pH adjusted to 6.83 by 3 N sulfuric acid solution; absorbance OD₅₅₀ was 0.3, 0.26 respectively; no sludge was produced after settling for 40 minutes.
3. Original wastewater with pH adjusted to 6.64 by 3 N sulfuric acid solution and 5 ml of 0.05% chemical organic flocculant EA-630 (Jiu-He International) was added; absorbance OD₅₅₀ was 0.29, 0.19 respectively; the settled-sludge volume was 1 ml, 4 ml respectively after settling for 40 minutes.
4. Original wastewater with pH adjusted to 6.74 by 5% aluminum chloride solution; absorbance OD₅₅₀ was 0.16, 0.17 respectively after settling for 40 minutes; the settled sludge volume was 8 ml, 7 ml respectively.
5. Original wastewater with pH adjusted to 6.74 by 5% aluminum chloride solution and 5 ml of 0.05% chemical organic flocculent EA-630 (Jiu-He International) was added; absorbance OD₅₅₀ was 0.13, 0.19 respectively after settling for 40 minutes; the settled sludge volume was 9 ml, 10 ml respectively.
6. Original wastewater with pH adjusted to 6.74 by 5% aluminum chloride solution and 1 ml of 0.05% chemical organic flocculent EA-630 (Jiu-He International) was added; absorbance OD₅₅₅₀ was 0.15, 0.15 respectively after settling for 40 minutes; the settled sludge volume was 8 ml, 8 ml respectively.

The powdered biological flocculants were applied by mixing the powders with tap water into a concentration of 1% powder solution. The flocculating activity was evaluated by mixing an appropriate amount of the bacterial solution and the powder solution, respectively, with 45 ml of the textile dyeing wastewater (Ho-Yo) and 0.25 ml of 5% poly aluminum chloride solution. The settled sludge volume and the absorbance (OD₅₅₀) of the supernatant were observed after keeping the mixture still for 30 minutes.

The comparison results of the flocculating activities for the powdered biological flocculent and the liquid biological flocculent (bacterial solution) are listed in Table 17. The wastewater from the textile dyeing industries that was treated by the 1% strain Y2105-1 powder solution leads to less sludge production but higher absorbance OD₅₅₀ for the supernatant, when compared with the wastewater that is treated by the bacterial solution. It is probably because that the suspended particles in the wastewaters are not completely removed. If the dosage of the powder solution is increased, the flocculating effects are reduced, thus decreasing sludge production and increasing absorbance OD₅₅₀ for the supernatant. However, the wastewater from the textile dyeing industries that is treated by the 1% strain K0214 powder solution leads to less sludge production and the lower absorbance OD₅₅₀ for the supernatant, when compared with the wastewater that is treated by the bacterial solution. As a whole, the flocculating activities of the biological flocculants toward the wastewater from the textile dyeing industries are affected by spray drying treatment.

TABLE 17
Comparison of the flocculating activities of the powdered
biological flocculant and the liquid biological flocculant

| Producing | | Bacterial solution | | 1% powder solution | |
|---|---|---|---|---|---|
| strain | Addition amount | SSV (mL) | OD₅₅₀ | SSV (mL) | OD₅₅₀ |
| Y2105-1 | 1 mL | 9.5 | 0.16 | 7 | 0.36 |
| | 2 mL | | | 6 | 0.4 |
| | 5 mL | | | 4 | 1.08 |
| K0214 | 1 mL | 12 | 0.64 | 7 | 0.26 |
| | 2 mL | | | 8 | 0.36 |
| | 5 mL | | | 5 | 0.97 |

### Example 9

### Application in Food Industry Treatment Plants

This experiment simulated the real operation situations in treatment plants, in particular, treatment plants in the food industry. In the early stage of screening the biological flocculant producing bacterial strains, the evaluation of their flocculating activities is carried out by simple flocculation test in 50 ml graduates. However, the treatment plants employ jar tests to determine the usage amount of the flocculants. Therefore, to simulate real treatment plant operations, this experiment employed the jar test to compare the flocculating capabilities of the biological flocculants and the commercialized organic flocculants.

The jar test used the Jar Tester (Shin-Kwan Precision Mechanics) to simulate the coagulation unit and the flocculation unit in the treatment plants, and the basic steps included:
1. After introducing 1000 ml of wastewater into the 1.0 L beaker, the Jar Tester was activated with the stirring speed set to 80 rpm and the solution was adjusted to an appropriate pH by an acidic solution or a basic solution.
2. Appropriate amount of the coagulant and the 5% aluminum chloride (AlCl_{3.6}H₂O) solution were added into the wastewater and the mixture was rapidly stirred for 3 minutes.
3. Appropriate amount of the flocculants, such as the biological flocculants or the chemical organic flocculants EA-630 (Jiu-He International) of the concentration 0.05% was added into the wastewater which undergone 30 seconds of flocculation.
4. The stirring speed was adjusted to 40 rpm, keeping the wastewater stirring for 15 minutes.
5. After stirring is stopped and the wastewater is kept still for 40 minutes, the chemical properties of the supernatant were analyzed and the settled sludge volume thereof was recorded.

A modified jar test was employed in this study. The usage amount of the wastewater in the jar test was originally set to be 1000 ml. However, the tested amount of the wastewater in each jar test was modified to be 500 ml for retrenching the usage amount of the wastewater. Because the indication of the beaker was in a scale of 100 ml, the experimental vessel was changed from 1.0 L beaker to the 500 ml trigone beaker having the indication in a scale of 10 ml, for accurately measuring the settled sludge volume.

Then, prototype powders of strain Y2105-1 were used in the coagulation treatment of the wastewater from the tofu fabrication process (Heng-E).

Biological flocculant application models that are similar to the application models of the chemical flocculants were established. The method for evaluating the operation conditions and the addition amount of the chemical flocculants were applied in the tests to determine the dosage of the biological flocculants and the practicability of the operation conditions for the biological flocculants. Usually, for the application of chemical flocculants in the plants, the application dosage and the operation conditions are established based on the following steps:
1. The dosages of the coagulant and the flocculants are fixed, and the most suitable operation pH value is determined.
2. By using the most suitable operation pH value, the most suitable dosage of the coagulant is verified.
3. By using the most suitable operation pH value and the most suitable dosage of the coagulant, the most suitable dosage of the flocculants is verified.

The dosage of 1% Y2105-1 powder solution was fixed to 1.0 ml, the best operation pH ranges from 6.3 to 6.5 (Table 18), when the dosage of 5% aluminum chloride (PACl) was 2 ml.

In comparing the influences on the flocculating capabilities under various dosage of the aluminum chloride solution (Table 19), the application of the flocculants increased the removal rate of the suspended solids in the wastewater. Regardless of whether 0.5 ml of 0.05% EA-630 solution or 1.0 ml of 1% Y2105-1 powder solution was added, the absorbance OD₅₅₀ was lower than 0.15. The addition of flocculants also increased the density of the settled sludge and thus reduced the settled sludge volume. However, the sludge amount resulting from the treatment of the biological flocculants was higher, about 20 ml˜60 ml more than that from the treatment of the chemical flocculants.

The differences between the application of the biological flocculants and the chemical flocculants in the most suitable dosage for treating the wastewater from the tofu fabrication process are listed in Table 20. For both the biological flocculants and the chemical flocculants, the settled sludge volumes were lower than 100 ml, with their absorbance OD₅₅₀ lower than 0.05. After the treatment, the COD values of both were between 300 mg/L to 400mg/L, with no great differences.

TABLE 18
The influence of pH values on the treatment of the Y2105-1 powders for
the wastewater from the tofu fabrication process

| | | | | | | |
|---|---|---|---|---|---|---|
| The starting pH value | 6 | 7.5 | 8 | 8.5 | 9 | 9.5 |
| After reaction | 6 | 6.3 | 6.85 | 6.5 | ND | ND |
| pH value | | | | | | |
| SV(mL) | 0 | 110 | 95 | 100 | ND | ND |
| OD₅₅₀ | 0.453 | 0.148 | 0.157 | 0.145 | ND | ND |

Note:
The addition amount of 1% Y2105-1 powder solution was 1.0 ml, while the addition amount of 5% aluminum chloride was 2 ml.
ND means no data; because the turbidity of the supernatant was obviously higher than other treatment conditions, no measurement was made.

TABLE 19
The influence on the flocculating activities under various amounts
of the aluminum chloride solution

| | Control | | 0.05% polymer | | 1% Y2105-1 | |
|---|---|---|---|---|---|---|
| 5% PACI | (no flocculant) | | (0.5 mL) | | (1 mL) | |
| usage amount | SSV | | SSV | | SSV | |
| (mL) | (mL) | OD₅₅₀ | (mL) | OD₅₅₀ | (mL) | OD₅₅₀ |
| 0.25 | 220 | 0.161 | 75 | 0.147 | 130 | 0.12 |
| 0.5 | 215 | 0.113 | 80 | 0.096 | 140 | 0.085 |
| 1 | 190 | 0.064 | 80 | 0.047 | 120 | 0.044 |
| 2 | 150 | 0.031 | 80 | 0.032 | 110 | 0.019 |
| 3 | 170 | 0.023 | 10 | 0.029 | 130 | 0.018 |
| 4 | 190 | 0.025 | 100 | 0.026 | 120 | 0.015 |
| Water pH = 6.3 | 80 | 0.182 | | | | |

TABLE 20
The treatment results by using the biological flocculant and the
chemical flocculant for treating the wastewater from the tofu
fabrication process

| Coagulant | Flocculant | | | |
|---|---|---|---|---|
| 5% PACI | 0.05% EA-630 | SSV | OD₅₅₀ | COD |
| 1 mL | 0 mL | 80 mL | 0.028 | 320 mg/L |
| | 0.1 mL | 95 mL | 0.029 | 375 mg/L |
| | 0.5 mL | 90 mL | 0.031 | 350 mg/L |
| | 1 mL | 90 mL | 0.025 | 300 mg/L |
| 2 mL | 0 mL | 80 mL | 0.018 | 400 mg/L |
| | 0.1 mL | 95 mL | 0.02 | 730 mg/L |
| | 0.5 mL | 90 mL | 0.022 | 315 mg/L |
| | 1 mL | 90 mL | 0.025 | 330 mg/L |

| Coagulant 5% | Flocculant | | | |
|---|---|---|---|---|
| PACI ( mL) | 1% Y2105-1 (mL) | SSV | OD₅₅₀ | COD |
| 1 mL | 0 mL | 75 mL | 0.034 | 315 mg/L |
| | 0.5 mL | 80 mL | 0.046 | 315 mg/L |
| | 1 mL | 80 mL | 0.042 | 385 mg/L |
| | 2 mL | 80 mL | 0.029 | 280 mg/L |
| 2 mL | 0 mL | 80 mL | 0.029 | 275 mg/L |
| | 0.5 mL | 90 mL | 0.022 | 275 mg/L |
| | 1 mL | 90 mL | 0.032 | 330 mg/L |
| | 2 mL | 85 mL | 0.029 | 360 mg/L |
| Water PH 6.53 | 0 | 60 mL | 0.162 | 555 mg/L |

Note:
the operation pH value was 6.4.

Therefore, biological flocculant of strain Y2105-1 was used in jar test to simulate real plant operation, and application of biological flocculant was based on the application method of chemical flocculant in real plant operation.

### Example 10

### Activation of Bacterial Strains

In the process of producing biological flocculants, biological flocculant producing bacteria are identified, frozen and preserved in −70° C. and subsequently activated and verified for production. The main objective of activation is to boost cultivation of the bacterial strains in the proper stage, which serves as the source of seed bacteria for the main fermentation step. The activating conditions should be helpful for the bacteria to make contact with and to absorb nutrients, without growth inhibitory factors, thus enhancing the growth and proliferation of microbes and obtaining the activated bacterial strain with stable activity, at the proper stage, and in uniform morphology.

Various activating culture media were studied in this experiment. The biological flocculant producing bacteria Bacillus endophyticus (Y2105-1) was activated using solid culture, and the activating culture medium compositions for all the biological flocculant producing bacteria tested in this experiment are listed in Table 21. The activating procedure for the biological flocculant producing bacterial strain was to inoculate the bacterial strains onto the solid culture media listed in Table 21 after thawing the preserved bacterial strain under room temperature and to incubate the inoculated culture media for 2˜3 days under 30° C. The guideline for selecting the activating culture media was that the bacterial colonies grown on the culture media should have strong activities helpful for subsequent seed bacteria culture cultivation. Therefore, if the morphology of colonies grown on one particular culture medium was irregular, indicating varying growth of the colonies, this particular culture medium should not be used as the activating culture medium. The growth rates of the preserved bacterial strain in three activating culture media were similar. However, because the morphology of the bacterial colonies cultivated with TSA medium was more uniform, it was more suitable to use TSA medium as the activating culture medium for the bacterial strain.

For production purpose, the procedure for activating the preserved bacterial strain is to streak the preserved bacterial strain onto the TSA plate culture medium through the sterile platinum spatula in the sterilized hood and incubate the plate culture medium for 1-2 days under 30° C. The producing bacterial strain in TSA plate culture medium can be preserved for 7 days for use in the seed bacterial culture.

TABLE 21
The composition formulation of the activating culture medium for the
biological flocculants producing bacterial strains

| Formula designation | Composition formulation | Concentration |
|---|---|---|
| PDA | Potato dextrose agar (Difco) | 39 g/L |
| NA | Nutrient broth agar (Difco) | 20 g/L |
| TSA | Tryptic soy agar (Difco) | 40 g/L |

### Example 11

### Preparation of Seed Bacteria Culture

Seed bacteria culture was prepared in this experiment. Seed bacteria culture is used to produce large amounts of bacteria cells for fermentation production. Therefore, along with the nutrients essential for bacterial growth, the seed culture medium includes the readily convertible carbon and nitrogen sources to satisfy the nutrition requirements for bacterial growth in order to obtain active bacteria in abundance for inoculation in the main fermentation tank, thus increasing the cultivation efficiency of the main fermentation.

The light microscope (Nikon, model AFX-2A) with the magnification of 400 times (400×) or 1000 times (1000×) can be used to observe the growth of the biological flocculant producing strains. The observation is focused on checking the growth of the producing microbial strain and whether the culture is polluted by other non-producing microbial strains.

The growth of the biological flocculant producing strains in the culture medium can also be measured by changes in the absorbance (optical density) of the culture medium under visible light wavelength 600 nm (OD₆₀₀). The measurement of the absorbance is as follows: diluting the culture medium with de-ionized water until the absorbance is between 0.1-0.3, using de-ionized water as the blank control, and measuring the absorbance of the diluted culture medium under wavelength 600 nm according to the measurement procedure of the spectrophotometer (DU-50, Beckman). The bacterial cell concentration is the product of the absorbance value of the diluted culture medium under wavelength 600 nm multiplying the dilution times, shown in OD₆₀₀.

In the experiment, the method of culturing seed bacteria for the biological flocculent producing bacteria was performed as follows:
1. The colonies grown on the TSA activating culture medium were scratched and incubated with 5 ml of seed culture solution.
2. The 5 ml seed culture solution with the activated bacterial strain was added to another 200 ml of seed culture solution in the 500 ml shaker and the mixture was incubated under 30° C., shaking in 160 rpm, for 24 hours. The culture solution used was the same culture solution developed in Example 12.
3. A sample was taken from the mixture. The sample was observed under the microscope to observe the growth of the microbes and its absorbance (OD₆₀₀) was measured.

### Example 12

### Preparation of Production Culture Medium

In this experiment, appropriate production culture media are studied. Good-quality production culture media should have properties including (1) high yields of the target products, (2) healthy growth of the bacteria cells with short fermentation periods, (3) low-cost culture media having stable sources of raw materials and (4) easy recovery of the target products. Usually, the growth of the microbes and the accumulation of their metabolic products are closely related. Therefore, when considering the compositions of the production culture medium, it is necessary to simultaneously take into account speeding up the growth and accelerating accumulation and production of the metabolic products.

The major constituents of a bacterial cell include carbon, nitrogen, phosphor, sulfur, aluminum and others, while carbon accounts for almost 50% of the total dry weight of the cell and nitrogen accounts for 7˜12%. Carbon source provides the energy required for vitality of the bacterial strain. It is the source for bacterial cell constituents and metabolic products. It is also the major nutrients in the liquid bacterial culture. The common carbon source include:
1. monosaccharides: such as glucose;
2. disaccharides: such as maltose, lactose and sucrose etc.;
3. polysaccharides: such as sugar molasses, corn starch and potato starch;
4. fats.
   Except for monosaccharides, the other carbon sources need to be digested into monosaccharides by the enzymes produced from the bacterial cells for utilization. The nitrogen source is mainly used as the source for bacterial cell constituents and nitrogen-containing metabolites. The common nitrogen source can be divided into two types: the organic nitrogen source and the inorganic nitrogen source. The organic nitrogen source include, for example, soybean powder, peptone, yeast powder, yeast extract, fish powder, blastema powder, rice bran hydrolytic solution, soybean protein, soybean steep liquid and corn steep liquid etc. The inorganic nitrogen source include ammonium chloride and urea etc. In addition to an abundance of proteins, polypeptides and free amino acids, the organic nitrogen source usually also contains small amount of sugar, fats, microelements and vitamins, thus satisfying the basic requirements of the bacteria in a well-balanced manner. In general, organic nitrogen is more suitable for the bacterial growth than inorganic nitrogen.

a. The Studies of the Carbon Source

Tests were carried out to determine the most suitable carbon source. Choices were made between recipes of carbohydrates including corn starch, glucose, sucrose and sugar molasses etc. Also the culture medium was added with appropriate amounts of yeast extract or nutrient broth for extending and increasing the utilization of the carbon source and providing complete nutritious compositions in promoting the secretion of the active materials. The testing method included using individual carbohydrate composition to prepare 200 ml of the culture medium in the 500 ml shaker, autoclaving the medium, inoculating the producing bacterial strain to the autoclaved medium, and incubating the medium in the shaking incubator under 30° C. and 160 rpm for 4 days. Then, during the experiment, microbes were sampled to observe their growth condition, and the pH value and the flocculating activities of the fermentation solution were measured. After comparing the test results for the utilization of the different carbohydrates, it was concluded that the production culture medium mainly consisting of glucose is most suitable for the growth of the biological flocculants producing bacteria.

b. The Studies of the Nitrogen Source

Tests were carried out to determine the most suitable nitrogen source. Selections were made between the organic nitrogen sources, including nutrient broth, peptone, hydrolytic soybean protein and yeast extract etc. and the inorganic nitrogen sources, including ammonium sulfate and ammonium chloride etc., together with the culture medium containing 15 g/L glucose. The choice of the culture medium was based on the flocculating activities. Based on the experimental results, the combination of yeast extract and glucose is most suitable for the growth of the producing bacteria.

c. The Effects of Inorganic Salts

Microbes have smaller demands for inorganic salts than for carbon and nitrogen sources, but the concentration and the varieties of the inorganic salts have decisive influences on the metabolism of the microbes. For the inorganic salt recipes of the culture medium, the concentrations of phosphor and aluminum are higher. Phosphor is the main compositional ingredient for the nucleic acids, the nuclear proteins, phospholipids and many phosphate functional groups of coenzymes. Aluminum can stabilize the nuclear proteins, the cellular membrane and the nucleic acids and is an activating agent for certain enzymes. Therefore, both elements are essential materials for bacterial liquid fermentation.

This experiment tested the effect of adding inorganic salts by utilizing potassium bihydrogen phosphate (KH₂PO₄) and magnesium sulfate (MgSO₄), and the influences of the inorganic salts on the bacterial growth and the flocculating activities of the bacterial solution were observed. After testing various recipes of phosphate salts and potassium salts, the results showed that the addition of inorganic salts produced no obvious benefits to the growth of the biological flocculant producing bacteria and the flocculating activities of the bacterial solution. As for other inorganic salts such as calcium, sodium, sulfur, iron, zinc, manganese, cobalt and copper etc., this experiment used molasses as the source for all these microelements because their required quantities are low and high concentrations of these microelements sometimes inhibit the bacterial growth.

From the above testing results of the culture medium, it was concluded that the preliminary recipe of the fermentation culture medium includes the carbon source, consisting mainly of glucose and molasses, and the nitrogen source of hydrolytic soybean protein. This production culture medium is termed as GSM.

### Example 13

### Conditions Suitable for Fermentation

Assessments for determining the most suitable conditions of fermentation production included (1) improvements of the production culture medium,

(2) adjustments of cultivation conditions including temperature, ventilation volume (VVM) and pH values etc., and (3) establishment of culture time (days) etc. The testing method involved carrying out fermentation production in a 5 L or 7 L fermentation tank (Mitsuwa) using various production culture media with the inoculation ratio of 2%˜5% of seed culture solution. The incubation temperature was set at 28˜30° C., with fermentation time of 4 days. During the fermentation process, the changes in pH values (Suntex, microprocessor 2000A), dissolved oxygen concentrations and air flow volumes were recorded, and the rotation speed of the stirrer and ventilation volume were adjusted depending on the requirements, together with taking samples periodically to monitor the growth. The monitoring items of the bacterial growth include (1) microscopy observation of the growth wherein the method is as described in Example 11, (2) glucose concentration analysis, (3) determination of the total dry weight percentage for the fermentation solution, and (4) examination of the flocculating activities of the bacterial solution.

Analysis of glucose concentration is explained in detail below. Glucose is the major ingredient of the carbon source in the culture medium and changes in glucose concentration during the culture process can be used as a monitoring index for the fermentation and growth of the bacteria. Usually the culture medium needs to have enough glucose for satisfying the requirements of high bacterial production. However, very high glucose concentration (>50%) may inhibit the growth of bacteria cells. The measurement of glucose was performed by high performance liquid chromatography (HPC) analyzed by refractometer (Shimadzu, RID-10A). The HPLC operation conditions were RP-18 column (Gilson), column temperature 65° C. (Bio-rad column heater), column flow rate 0.6 ml/min (Shimadzu, LC-1 OAT vp), mobile phase

0.002 N H₂SO₄. The sample pretreatment involved centrifuging (9000 rpm, 3 minutes,

4° C.; Eppendorf 5415C) the fermentation bacterial solution to remove the bacterial cells, followed by diluting with distilled water to 20 times for subsequent measurement.

Changes in pH values were also studied. During fermentation, the pH value of culture medium GSM kept decreasing following the metabolism of the carbon source. After cultivation for 4 days, its pH value was reduced to 5.5. Afterwards, the pH value gradually increased due to the production of numerous metabolites. At last, the pH value was 8.5, as nutrition was exhausted, spores were formed and the bacterial strain was aging, leading to automatic cell lysis. The biological flocculent producing bacteria endured a wide pH range and its growth was not affected during the pH 5˜8. Therefore, only during the preparation of the culture medium, the pH of the culture medium was adjusted to 7 and no further adjustment was required during the fermentation period owing to the pH varying between 5˜7 during the fermentation period.

Ventilation condition for the culture media was also studied. Because the biological flocculant producing bacteria grow aerobically, insufficient supply of dissolved oxygen during the cultivation will inhibit the production of bacterial cells and influence the generation of metabolic products. During the fermentation period, the ventilation volume of the airflow should be maintained above 0.5 VVM (volume/air flow volume/minute). But bubbles will form to affect the normal operation of the fermentation tank if too high a ventilation volume is provided. After many tests, according to the changes in the total dry weight percentage of the fermentation solution, when the ventilation volume of the airflow during fermentation was within the range of 0.5˜1.0 VVM and the stirring speed was between 200˜300 rpm, the fermentation time can be shortened from 88 hours to 40˜48 hours.

The method for determining the total dry weight is as follows. The total dry weight percentage is the total weight of the remained solid from the 100 grams of the cultivation solution or bacterial fermentation solution after drying, shown in weight percentage (% w/w). The value is usually used to evaluate the recoverable solid amounts in the bacterial fermentation solution. The main sources of the solids from the culture medium and the fermentation solution are bacterial cells, microbial metabolites and the remained medium. In general, before the fermentation of the culture medium the total dry weight of the culture medium is highest. However, the total dry weight of the culture medium decreases as the culture medium turns into the energy of microbial growth, bacterial cells and metabolites. The method for measuring the total dry weight percentage is to dry-up the culture medium or the fermentation solution in 120° C. oven until its weight reaches constant and calculate the percentage of the remained dry weight to the original weight of the sample.

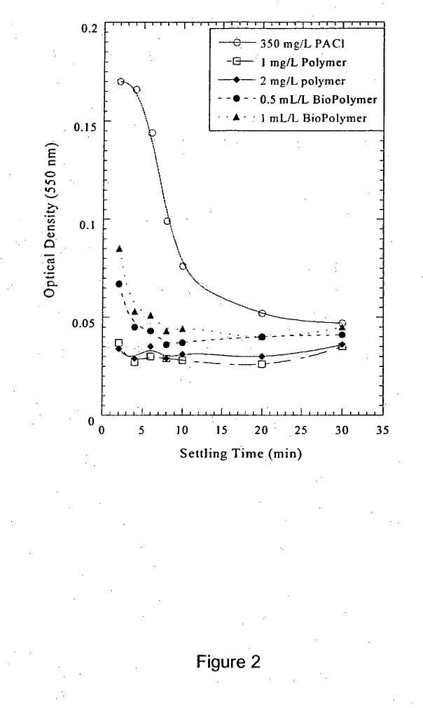

The flocculating capabilities of the fermentation bacterial solution obtained from the culture medium GSM with different culture time and the chemical flocculants toward the textile dyeing wastewater (Shin-Long) were compared and shown in FIG. 2. From the absorbance (OD₅₅₀) changes of the supernatant, an appropriate amount of coagulant was added. After 30 minutes of the settling time, no obvious difference was observed between the biological flocculants and the chemical flocculants. In the early stage of the settlement, significant changes were observed in the absorbance (OD₅₅₀) of the supernatant in the first five minutes of settling due to the properties of the formed floc density and the settling speed etcAs the absorbance for unreated water, whether it's kaolin suspensions or industrial wastewater, will vary depending on the time differences in preparing, acquiring, storing or experimenting the samples, therefore, the reduction in the absorbance (OD₅₅₀) of the supernatant in the first five minutes of settling was used as an evaluation criterion for comparing the flocculating activities of the bacterial solutions.

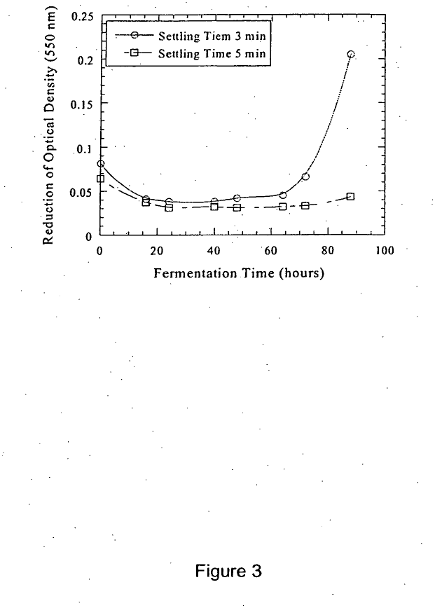

The flocculating activities of the bacterial solution, cultivated with the culture medium GSM, vary along with the fermentation time, as shown in FIG. 3. After uniformly mixing the textile dyeing wastewater (Shin-Long) with the bacterial solution, the reduction of the absorbance (OD₅₅₀) of the supernatant following standing for three minutes or five minutes was used to evaluate the flocculating activities of the bacterial solutions. If the fermentation time was less than 60 hours, no obvious absorbance reduction was observed, indicating that the obtained bacterial solution had no evident flocculating effects on the textile dyeing wastewater (Shin-Long). If the fermentation time was longer than 88 hours, absorbance reduction kept increasing, indicating that the bacterial solution had flocculating capabilities on the textile dyeing wastewater (Shin-Long).

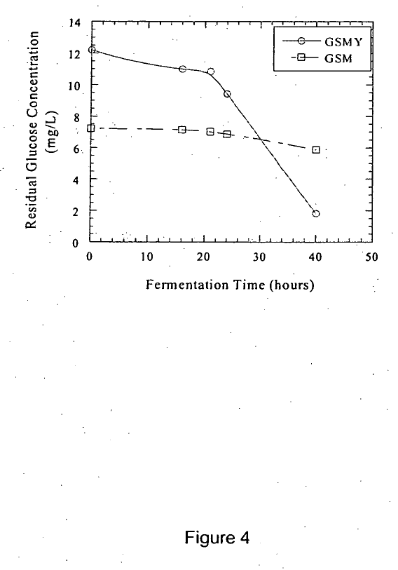

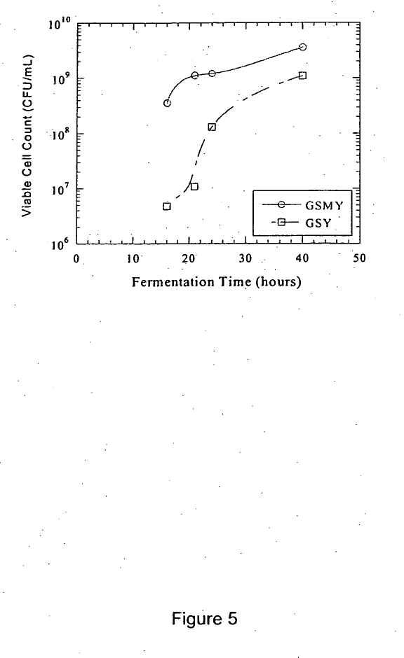

The culture medium was modified by adding yeast extracts to stimulate the preliminary growth of the biological flocculants producing bacteria, and this modified culture medium is designated as GSMY. FIG. 4 shows the comparison of the glucose metabolic rates for the culture media GSM and GSMY during the fermentation processes. The production culture medium GSMY added with yeast extracts had a faster metabolic rate for glucose. FIG. 5 shows the comparison of the total bacteria count (CFU/ml) for the culture media GSM and GSMY during the fermentation processes. The bacteria grown on the production culture medium GSMY added with yeast extracts has a faster growth rate. In conclusion, the yeast extracts are more suitable for the preliminary growth of the biological flocculant producing bacteria, when compared with hydrolytic soybean protein.

The total bacteria count is the fundamental parameter for evaluating the cultivation of the microbes, by using the solid plate culture medium for directly measuring the bacteria count per unit volume, thus evaluating the proliferation of the bacteria cells during fermentation cultivation. The method for measuring the total bacteria count is as follows: diluting 1 ml of fermentation solution into a series of concentrations, streaking-0.1 ml of dilutions from various concentrations onto the suitable plate culture medium (Tryptic Soy Agar, TSA, Difco), placing the culture plate into the 30° C. incubator for 24 hours, and counting the colony forming units (CFU) in every culture plate after 24 hour incubation. The potent measuring samples have the colony forming units between 30-300. The average value of the products from multiplying CFU with the dilution time for various potent samples is the total bacteria count, shown in the unit of CFU/ml.

Taking into account the flocculating capabilities of the fermentation solution, obtained from the fermentation processes, toward the textile dyeing wastewater (Shin-Long) as a basis for the modification of the culture medium, the production culture medium GSMY of the biological flocculant producing bacteria includes glucose, hydrolytic soybean protein, molasses and yeast extracts. In the early stage fermentation, the producing bacterial strain digests the yeast extracts to generate biomass, and uses the produced enzymes to decompose the soybean protein into easily absorbed small molecules for further development.

As concluded from the above experimental results, the most suitable fermentation culture medium and the most suitable fermentation conditions are as follows:
1. The production culture medium GSMY consists of glucose, hydrolytic soybean protein, molasses and yeast extracts.
2. The most suitable temperature for the fermentation culture is between 28-30° C., because the temperature lower than 28° C. results in slow growth and a longer fermentation period.
3. The fermentation time is 40-48 hours.
4. The ventilation volume of the airflow is 0.5-1.0 VVM.
5. No pH adjustment is required during the fermentation period, and simply adjusting the pH value of the culture medium to 7.0 in preparing the culture medium.

### Example 14

### Fermentation Strategy

Strategies can be designed for fermentation. According to the analysis results on changes in glucose concentration during the fermentation processes, glucose still exists in the fermentation solution after 16˜24 hours of fermentation. However, the bacterial cell concentration will not keep increasing if the fermentation time is extended, possibly due to insufficient supply or unbalance of nutrients. In order to save the fermentation costs, the most appropriate strategy is to create the highest production of the bacterial cells without nutrient remnants after completing the fermentation.

The strategy of using the culture medium GSM as the starting culture medium in fermentation production and subsequent addition of glucose in batches into the culture medium was evaluated. The addition of glucose was performed by adding 50 grams of glucose (dissolved in 200 ml de-ionized water) to the fermentation tank on the second day of fermentation, and the residual amount of glucose and the flocculating activities of the bacterial solution were observed on the fourth day of fermentation. The results showed that glucose were not completely consumed in this manner of fermentation and the stability of the flocculating activities for the product was not good, and the variation of the flocculating activities for different fermentation batches was huge.

Then the evaluation of using the culture medium GSMY as the production culture medium was performed. It was shown that the culture medium containing the yeast extracts can satisfy the nutrition requirements for the continuous growth of the bacterial strain that adapts to the new environment. Thus, the growth lag phase was shortened and the production time was decreased from 4 days to 2 days. When the culture medium GSM was used as the production culture medium, the control of pH values had no notable influences on the fermentation quality. When the culture medium GSMY was used as the production culture medium, the flocculating activity of the fermentation solution was elevated. However, besides incomplete utilization of the culture medium, the pH value of the fermentation solution was reduced to below 5, so that pH adjustment was required for the fermentation process. As shown in FIGS. 4 and 5, for the culture medium (GSMY) added with the yeast extracts, the glucose consumption rate was increased, and after 16 hour culture, the number of the alive bacterial cells therein was about twice of that of the alive bacterial cells in the culture medium GSM (without adding the yeast extracts), thus shortening the adaptation period of the bacterial strain.

The results of jar tests by using GSM fermentation solution and GSMY fermentation solution to treat the textile dyeing wastewater (Shin-Long) are listed in Tables 22 and 23. After adding 350 mg/L control aluminum chloride (AlCl₃) solution into the untreated wastewater, the mixture was adjusted to have the pH value of 6.97 and 6.72 respectively as the standard testing solution and the control for this flocculation experiment. Each individual sample was added to the standard testing solution for performing the jar test. The settled sludge volume (SSV) in various setting time was recorded and the sludge settling speeds after standing for 2 minutes and 3 minutes were compared. The results showed that the fermentation solution from the culture medium GSMY containing the yeast extract (Table 23) results in less settled sludge volume and has a sludge settling speed faster than that of the commercialized chemical organic flocculants (EA-630, Jiu-He International). Furthermore, its chemical oxygen demand (COD) is reduced from 1640 mg/L to 300 mg/L, comparable to the treatment effects of the organic chemical flocculants.

Chemical oxygen demand (COD, unit: mg/L) is one of the commonly used indexes for monitoring the organic compound concentration in the water. Also COD is one of the standard monitoring items of the effluent discharge criteria set by the Environmental Protection Administration. Taking the wastewater from the textile dyeing industries as an example, the discharge criterion of COD for the effluent is 100 mg/L. The method for measuring COD is carried out by using potassium dichromate under the catalysis of concentrate sulfuric acid and high temperature (150° C.) to oxidize the organic compounds in water, and after the reaction is completed, measuring the concentration of remaining potassium dichromate by colorimetry (Hack DR-2000 Spectrophotometer) to obtain the chemical oxygen demand of the water sample.

Therefore, GSMY fermentation solution has comparable COD and smaller SSV values than those of the chemical organic flocculants.

TABLE 22
The evaluation of the flocculating capacities of the fermentation
bacterial solution in the culture medium GSM toward
the wastewater from the textile dyeing industries

| | SSV (mL/500 mL)^{#} | | | | |
|---|---|---|---|---|---|
| Flocculant type and dosage* | 2 min | 3 min | 5 min | 10 min | COD (mg/mL) |
| Untreated wastewater | | | | | 1640 |
| Control | 410 | 300 | 220 | 140 | 311 |
| Examples | | | | | |
| 1 mg/L organic flocculant EA-630 | 330 | 240 | 180 | 125 | 301 |
| 2 mL/L inoculating bacterial solution | 350 | 250 | 195 | 135 | 342 |
| 1 mL/L 16 hr fermentation bacterial solution | 275 | 200 | 155 | 127 | 326 |
| 1 mL/L 40 hr fermentation bacterial solution 1 | 285 | 210 | 170 | 128 | 289 |
| 1 mL/L 40 hr fermentation bacterial solution 2 | 320 | 230 | 185 | 135 | 296 |
| *the source of the untreated wastewater was the Shin-Long textile dyeing factory, the testing solution was obtained from adding 350 mg/L PACI into the untreated wastewater and adjusting the pH value equivalent to 6.97. The types and dosages of the flocculants listed under the Examples in the above table are | | | | | |
| # based on the added amount of the flocculants into the testing solution. EA-630 was provided by Jiu-He International Co.. | | | | | |

^{#}SSV = settled sludge volume.

TABLE 23
The evaluation of the flocculating capacities of the fermentation
bacterial solution in the culture medium GSMY toward the
wastewater from the textile dyeing industries

| | SSV (mL/500 mL)^{#} | | | |
|---|---|---|---|---|
| Flocculant type and dosage* | 2 min | 3 min | 5 min | 10 min |
| Control | 450 | 350 | 185 | 100 |
| Examples | | | | |
| 1 mg/L organic flocculant EA-630 | 350 | 320 | 130 | 80 |
| 1 mL/L 40 hr fermentation bacterial | 230 | 170 | 120 | 85 |
| solution 1 | | | | |
| 1 mL/L 40 hr fermentation bacterial | 200 | 150 | 110 | 80 |
| solution 2 | | | | |
| 1 mL/L 40 hr fermentation bacterial | 200 | 155 | 116 | 83 |
| solution 3 | | | | |

*the source of the untreated wastewater was the Shin-Long textile dyeing factory, the testing solution was obtained from adding 350 mg/L PACI into the untreated wastewater and adjusting the pH value equivalent to 6.72. The types and dosages of the flocculants listed under the Examples in the above table are based on the added amount of the flocculants into the testing solution. EA-630 was provided by Jiu-He International Co..
^{#}SSV = settled sludge volume.
Note:
after adding PACI 350 mg/L, the pH value was adjusted to 6.72.

### Example 15

### Dosage of the Biological Flocculants

The appropriate amount of biological flocculants to be used is studied. In order to find out the best usage amount (dosage) for the biological flocculants, the textile dyeing wastewater (Shin-Long) was used to evaluate the flocculating effects for different dosages of the fermentation solution with 2 mg/L chemical flocculants (EA-630, Jiu-He International) added. The method for preparing the testing solution for the jar test is to add 500 mg/L aluminum chloride solution into the untreated wastewater and adjust the pH value of the mixture to 6.57 with 3 N sulfuric acid or sodium hydroxide solution.

Due to variations in the water qualities of the wastewater, the most suitable usage amounts of the flocculants and the coagulating conditions differ for different types of wastewater. The jar test was employed to decide the dosages and operation conditions of the flocculants. The jar test uses the Jar Tester to simulate the coagulation and the flocculation units in the treatment plants, and the basic operating steps include:
1. After introducing 1000 ml of wastewater into the 1.0 L beaker, the Jar Tester (Shin-Kwan Precision Mechanics) is activated with the stirring speed being set to 80 rpm and the solution is adjusted to an appropriate pH by an acidic solution or a basic solution.
2. Appropriate amounts of the coagulant, such as 5% aluminum chloride (AlCl_{3.6}H₂O) solution are added into the wastewater and the mixture is rapidly stirred for 3 minutes.
3. An appropriate amount of the flocculant, such as the biological flocculant or the chemical organic flocculant EA-630 (Jiu-He International) of concentration 0.05%, is added into the wastewater for 30 seconds of flocculation.
4. The stirring speed is adjusted to 40 rpm and the wastewater is stirred for 15 minutes.
5. The stirring is stopped and the wastewater is kept still for 40 minutes. Thereafter, the chemical properties of the upper clear solution are analyzed and the settled sludge volume thereof is recorded.

During the settling process, the sizes, the formation rates and the settling rates of the flocs are observed at the same time, and the settled sludge volumes at different time points are recorded. Usually, as the density of the floc is higher, the settling speed is faster. On the other hand, when the density of the floc is lower, the settling speed is slower. The sludge is easily stirred by disturbance and easily lost, thus leading to inferior flocculating effects.

A modified jar test was used in this study. The usage amount of the wastewater in the jar test was originally set to be 1000 ml. However, the tested amount of the wastewater in each jar test was modified to 500 ml for retrenching the usage amount of the wastewater. Because the indication of the beaker was in a scale of 100 ml, the experimental vessel was changed from 1.0 L beaker to the 500 ml trigone beaker having the indication in a scale of 10 ml, for accurately measuring the settled sludge volume.

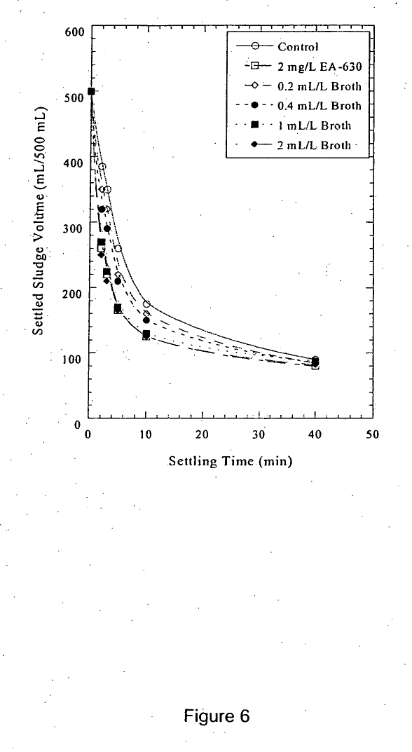

The results show comparable chemical and biological flocculant effects at certain dosage. As shown in FIG. 6, as the added amount of the fermentation solution to the testing solution was 2 mL/L (1 ml/500 ml), the settled sludge volumes of settling for 2 minutes and 3 minutes were similar to the settled sludge volume resulting from using the chemical organic flocculants (EA-630) with 2 mg/L dosage (addition amount). This indicates that the flocculating activities of the fermentation solution and the organic chemical flocculants were comparable. The analysis results for the water qualities of the supernatant standing for 40 minutes are listed in Table 24, showing COD being reduced from 1420 mg/L to about 400 mg/L. Using the absorbance (OD₅₅₀) as an evaluation parameter, the fermentation solution with 2 mL/L dosage had the flocculating activity of 22.5, comparable to the flocculating activity (23.9) of the chemical organic flocculants EA-630 with 2 mg/L dosage. The fermentation solution (2 mL/L) had the better true color reduction rate, when compared with the chemical organic flocculants (2 mg/L).

The standard kaolin suspension (kaolin, Riedel-de Haen) was employed as the standard testing solution for the flocculating activity analysis. Preparation of the standard kaolin suspension is carried out by stirring the kaolin suspension of 5 g/L concentration for 5 minutes and, after keeping still for 2 minutes, taking the supernatant as the standard testing solution for testing flocculating activities. This suspension can be preserved for 1 month under 4° C., but prior to testing, the suspension should be restored back to room temperature.

Because the kaolin can not fully simulate the properties of the suspended particles in the industrial wastewater, real industrial wastewater was also used to evaluate the differences between the flocculating activities of the flocculants in the process of developing the biological flocculants.

The method for measuring the flocculating activities is carried out by mixing appropriate amounts of cation coagulants, such as calcium chloride (CaCl₂), aluminum chloride (AlCl₃), ferric chloride (FeCl₃) or ferrous sulfate (FeSO₄), into 500 ml of standard kaolin suspension or industrial wastewater, adjusting the pH value, adding appropriate amounts of biological flocculants or chemical organic polymeric flocculants, stirring for 2 minutes, and, after keeping still for 30 minutes, measuring the absorbance OD₅₅₀ of the supernatant. The differences between the experiments and the control are caused by whether the biological flocculant or chemical organic polymeric flocculant is added or not. The absorbance OD₅₅₀ of the supernatants for the control and the experiments are compared and the flocculating activities is calculated based on the following formulations:
Flocculating Activity=1/OD_{550,S}−1/OD_{055, C}
OD_{550,S}=optical density of the experimental sample under wavelength 550 nm
OD_{550,C}=optical density of the control (reference) sample under wavelength 550 nm

After the water sample was clarified, true color unit of the water sample was measured. The method for measuring true color unit refers to the analysis method “The measuring method for true color of the water—ADMI method—NIEA W223.50B” published by the Environmental Protection Administration, Taiwan, ROC. After removing the suspended particles in, the water by filtering the water sample through a 0.45 micron membrane, the transparency of the water sample was measured under three wavelengths 590 nm, 540 nm and 438 nm by spectrophotometer (Jasco). Using the color formulations set by the Environmental Protection Administration, the true color value of the water sample can be calculated. The results are set out in Table 24.

TABLE 24
Comparison of the flocculating results by the fermentation
solution and the chemical flocculants

| | Settling | Settling | Settling | Settling | Settling |
|---|---|---|---|---|---|
| Flocculant | 3 min. | 5 min. | 3 min. | 3 min. | 3 min. |
| type and | Absorbance | Flocculating | COD | SS | True color |
| dosage | (OD₅₅₀) | activity | (mg/L) | (mg/L) | (ADMI) |
| Untreated | 0.321 | | 1420 | | |
| wastewater | | | | | |
| blank | 0.186 | 2.3 | 410 | 38 | 91 |
| 2 mg/L | 0.037 | 23.9 | 389 | 24 | 97 |
| EA-630 | | | | | |
| 0.1 mL broth | 0.048 | 17.7 | 412 | 8 | 97 |
| 0.2 mL broth | 0.047 | 18.2 | 437 | 16 | 101 |
| 0.5 mL broth | 0.042 | 20.7 | 441 | 22 | 108 |
| 1.0 mL broth | 0.039 | 2.25 | 426 | 26 | 75 |

Note:
1. the source of the untreated wastewater (500 ml)was the Shin-Long textile dyeing factory, the testing solution was obtained from adding 500 mg/L PACI into the untreated wastewater and adjusting the pH value equivalent to 6.42.
2. Flocculating Activity = 1/OD_{550,S}− 1/OD_{550, C} OD_{550,S} = optical density of the sample under wavelength 550 nm OD_{550, C} = optical density of the control under wavelength 550 nm
3. Broth stands for fermentation solution

### Example 16

### Use in Combination With Coagulants

The possibilities of using the biological flocculants or the chemical organic flocculants in combination with coagulants such as magnesium sulfate (MgSO₄), calcium chloride (CaCl₂) and ferric chloride (FeCl₃) etc. for treating the industrial wastewater were evaluated. The untreated wastewater from the tofu food manufacturing plant (Heng-E) was used as the standard testing solution and the experimental results are listed in Table 25. In treating the wastewater from the tofu food manufacturing plant (Heng-E) with the combination of either the biological flocculants (2 mL/L) or the chemical organic flocculants (2 mg/L) and ferric chloride (150 mg/L) or aluminum chloride (150 mg/L), any of the drug combinations was able to show efficacy in decreasing COD of the supernatant from 1230 mg/L to 300 mg/L following standing for 40 minutes. Because the ferric chloride solution showed a sorrel color, it is better to use aluminum chloride in the combination for assessing the true color reduction rate.

The biological flocculants developed by this center have comparable flocculating effects as the chemical organic flocculant in treating the wastewater from the tofu food manufacturing plant (Heng-E) or from the textile dyeing industry (Shin-Long). Moreover, the biological flocculants in combination with ferric chloride or aluminum chloride have the best flocculating effects.

TABLE 25
The evaluation of the flocculating activities of the biological flocculant
toward the Heng-E tofu wastewater.

| | Supernatant | | Supernatant |
|---|---|---|---|
| | Absorbance | Flocculating | COD |
| Flocculant type and dosage | (OD₅₅₀) | activity | (mg/L) |
| Untreated wastewater | 0.278 | | 1230 |
| AlCl₃ 150 mg/L | 0.072 | 10.292 | 293 |
| AlCl₃ 150 mg/L and 2 mg/L | 0.035 | 24.974 | 271 |
| EA-630 | | | |
| FeCl₃ 150 mg/L | 0.086 | 8.031 | 286 |
| FeCl₃ 150 mg/L and 2 mg/L | 0.045 | 18.625 | 346 |
| EA-630 | | | |
| FeCl₃ 150 mg/L and 1 mL/L | 0.041 | 20.793 | 305 |
| broth | | | |
| FeCl₃ 150 mg/L and 2 mL/L | 0.039 | 22.044 | 333 |
| broth | | | |

Note:
1. the testing solution was obtained from adding PACl or FeCl₃ into the untreated wastewater and adjusting the pH value equivalent to 6.32.
2. Flocculating Activity = 1/OD_{550,S} − 1/OD_{550,C}
OD_{550,S} = optical density of the sample under wavelength 550 nm
OD_{550,C} = optical density of the control under wavelength 550 nm

### Example 17

### The Influence of Biological Flocculants on Different Wastewater

Influences of biological flocculants toward wastewater from different sources were studied. Due to the diversity in industries, raw materials, products and manufacturing processes, the wastewater may include different types of ions and suspended particles. Also the flocculating capabilities of various flocculants can be influenced by the big differences in wastewater qualities. In order to evaluate the flocculating capabilities of the biological flocculants toward the wastewater from different sources, the wastewater from four textile dyeing factories Shan-Chi, Hong-Hor,

Hor-Yo and Wu-Li was used for the jar tests. Table 26 lists the flocculation test results for the wastewater from all of the sources. The flocculating capabilities of the biological flocculants toward the wastewater from different sources were equivalent or superior to that of the chemical flocculants originally used in those factories.

TABLE 26
The evaluation of the flocculating capacities toward various kinds of
wastewaters

| | | SSV | | |
|---|---|---|---|---|
| | Flocculant type | (mL/500 mL) | | |
| Wastewater source | and dosage | 3 min | 5 min | COD (mg/mL) |
| Heng-E | 2 mL/L broth | 145 | 125 | 333 |
| | 2 mg/L polymer | 145 | 126 | 346 |
| Shan-Chi | 2 mL/L broth | 160 | 100 | 496 |
| | 1 mg/L polymer | 220 | 110 | 512 |
| Hong-Hor | 2 mL/L broth | 105 | 85 | 130 |
| | 1 mg/L polymer | 110 | 103 | 86 |
| Hor-Yo | 2 mL/L broth | 82 | 75 | 225 |
| | 1 mg/L polymer | 82 | 75 | 201 |
| Wu-Li | 1 mL/L broth | 140 | 90 | 229 |
| | 1 mg/L polymer | 160 | 105 | 164 |
| Shin-Long | 1 mL/L broth | 145 | 120 | 301 |
| | 1 mg/L polymer | 240 | 180 | 301 |

Note:
Broth stands for the fermentation solution, and polymer represents EA-630.

### Example 18

### Formulation of Biological Flocculants

A detailed study of the appropriate composition of production culture medium was conducted. After evaluating the flocculating activities of a number of culture media, it was found that GSMY culture medium had higher flocculating capabilities. Therefore, every ingredient in the formula of GSMY culture medium was assessed to determine the ingredient having the greatest flocculating ability. The compositional ingredients of all of the culture media are listed in Table 27. The formulations were divided into two categories of sterilized and non-sterilized, and the textile dyeing wastewater (Shin-Long) was used for the analysis of the flocculating capability.

The test results of the flocculating capabilities of different formulations toward the textile dyeing wastewater (Shin-Long) are listed in Table 28. Comparing the settled sludge volumes after settling for 3 minutes, except for the flocculating capabilities of two formulations No. 6 and No. 7 being less affected by the heating reaction, the other formulations had shown better flocculating capabilities after treatment under high temperature and high pressure. Formulation No. 8 had a flocculating capability comparable to the control (1 mg/L EA-630).

Referring to Table 27 for the formulations No. 9-14, the influences of two types of thermal treatments—high pressure heating (121° C., 1.5 atm, 20 minutes) and stir heating (60° C., 40 minutes) on the flocculating activities were evaluated. Their flocculating activities were tested using the Shin-Long textile dyeing wastewater, and the results are listed in Table 29. Comparing the settled sludge volumes after settling for 5 minutes, these two thermal treatments had similar flocculating activities. The formulations containing high concentrations of the nitrogen sources had better flocculating activities, while glucose in high concentrations can weaken the flocculating capabilities. All the experimental samples had comparable purging effects for the COD value, reducing the supernatant COD value from 1390 mg/L to about 300 mg/L, and showed no noticeable difference in the removal rate of the suspended particles. As concluded from the experimental results, a high concentration of nitrogen source has the potential to promote the flocculating capabilities of the formulations, while glucose in high concentration will reduce the flocculating capabilities of the formulations.

The flocculating effect of the different nitrogen sources was studied. The results of the flocculating potentials of various nitrogen sources are listed in Table 30. The Shin-Long textile dyeing wastewater is used as the testing solution and the formulations were tested after being treated with the 121° C. thermal treatment for 10 minutes. From the results, it is noted that different nitrogen sources showed no evident differences in the flocculating effects.

TABLE 27
The composition formulation of the culture medium

| | | | Molasses | Yeast |
|---|---|---|---|---|
| Number | Glucose (g/L) | Soy Protein (g/L) | (g/L) | Extract (g/L) |
| 1 | 20 | 15 | 5 | 5 (Difico) |
| 2 | 20 | 15 | 5 | |
| 3 | | 15 | 5 | 5 (Difico) |
| 4 | | 15 | 5 | |
| 5 | | 15 | | 5 (Difico) |
| 6 | | | 5 | |
| 7 | | | 5 | 5 (Difico) |
| 8 | | 15 | | |
| 9 | 20 | 15 | 5 | 5 (Difico) |
| 10 | | 15 | | 5 (Industry) |
| 11 | | 15 | | 5 (Difico) |
| 12 | | 15 | | 5 (Industry) |
| 13 | | 100 | | |
| 14 | | 15 | | |

TABLE 28
The evaluation of the flocculating capabilities of different compositions
toward the wastewater from the textile dyeing industries

| | Flocculant type and dosage | | | SSV (mL/500 mL) | | | | |
|---|---|---|---|---|---|---|---|---|
| Groups | Type | Thermal treatment | Dosage | 2 min | 3 min | 5 min | 10 min | 40 min |
| Control 1 | | | | | 410 | 300 | 140 | 80 |
| Biological example 1 | 1 | A | 1 mL/L | | 370 | 250 | 132 | 78 |
| | 1 | B | 1 mL/L | | 310 | 180 | 118 | 70 |
| | 2 | A | 1 mL/L | | 370 | 240 | 125 | 78 |
| | 2 | B | 1 mL/L | | 330 | 190 | 120 | 70 |
| | 3 | A | 1 mL/L | | 360 | 235 | 130 | 80 |
| | 3 | B | 1 mL/L | | 340 | 200 | 120 | 70 |
| Control 2 | | | | 400 | 340 | 200 | 165 | 88 |
| Biological example 2 | 5 | A | 1 mL/L | 350 | 300 | 190 | 165 | 96 |
| | 5 | B | 1 mL/L | 310 | 250 | 170 | 150 | 90 |
| | 6 | A | 1 mL/L | 370 | 310 | 180 | 160 | 95 |
| | 6 | B | 1 mL/L | 350 | 315 | 185 | 160 | 95 |
| | 7 | A | 1 mL/L | 370 | 300 | 180 | 158 | 97 |
| | 7 | B | 1 mL/L | 350 | 300 | 180 | 160 | 95 |
| Chemical example 1 | EA- | | | 260 | 19 | 130 | 92 | 52 |
| | 630 | | | | | | | |
| Biological example 3 | 4 | A | 1 mL/L | 340 | 265 | 170 | 118 | 60 |
| | 4 | B | 1 mL/L | 330 | 200 | 140 | 100 | 56 |
| | 8 | A | 1 mL/L | 275 | 200 | 140 | 102 | 56 |
| | 8 | B | 1 mL/L | 265 | 195 | 138 | 100 | 58 |

Note:
1. The testing solution was prepared by adding 350 mg/L PACI into the textile dyeing wastewater (Shin-Long), and then adjusting the pH values to 6.42 using 3 N NaOH solution. This was the control.
2. The composition formulations refer to Table 27. The thermal treatment A was performed under high temperature and normal pressure (60° C., 1.0 atm), while the thermal treatment B was performed under high temperature and high pressure (121° C., 1.5 atm).
3. For the supernatants with similar turbidity, when the settled sludge volume (SSV) is smaller, the sludge settling speed is faster and the sludge concentration is higher.

TABLE 29
The flocculating capabilities of different composition formulations toward
the wastewater from the textile dyeing industries

| Flocculant type and dosage | | | | | | | | | |
|---|---|---|---|---|---|---|---|---|---|
| | Thermal | | SV (mL/500 mL) | | | | | COD | SS |
| Type | treatment | Dosage | 2 min | 3 min | 5 min | 10 min | 40 min | (mg/L) | (g/L) |
| Control | | | 475 | 420 | 310 | 145 | 75 | 267 | 0.47 |
| Chemical example 1 | | 1 mg/L | 460 | 380 | 280 | 116 | 60 | 253 | 0.46 |
| EA-630 | | | | | | | | | |
| Biological example 1 | B | 1 mL/L | 450 | 370 | 280 | 145 | 80 | 289 | 0.51 |
| Formulation 9 | | | | | | | | | |
| 10 | B | 1 mL/L | 390 | 310 | 205 | 133 | 76 | 272 | 0.56 |
| 11 | B | 1 mL/L | 390 | 310 | 200 | 130 | 73 | 275 | 0.60 |
| 12 | B | 1 mL/L | 390 | 320 | 215 | 140 | 76 | 268 | 0.69 |
| 13 | B | 1 mL/L | 350 | 265 | 190 | 132 | 75 | 312 | 0.83 |
| 14 | B | 1 mL/L | 370 | 290 | 200 | 135 | 72 | 271 | 0.64 |
| Control 2 | | | 470 | | 280 | 145 | 70 | 275 | 0.45 |
| Chemical example 2 | | 2 mg/L | 460 | | 175 | 107 | 59 | 262 | 0.52 |
| EA-630 | | | | | | | | | |
| Biological example 2 | A | 1 mL/L | 470 | — | 270 | 142 | 68 | 302 | 0.53 |
| Formulation 9 | | | | | | | | | |
| 10 | A | 1 mL/L | 450 | — | 175 | 126 | 72 | 277 | 0.54 |
| 11 | A | 1 mL/L | 450 | — | 220 | 140 | 66 | 267 | 0.55 |
| 12 | A | 1 mL/L | 350 | — | 155 | 114 | 60 | 273 | 0.47 |
| 13 | A | 1 mL/L | 230 | — | 116 | 92 | 53 | 295 | 0.55 |
| 14 | A | 1 mL/L | 400 | — | 200 | 135 | 64 | 275 | 0.48 |

Note:
1. The testing solution was prepared by adding 350 mg/L PACI into the textile dyeing wastewater (Shin-Long), and then adjusting the pH values to 6.57 (control 1) or 6.45 (control 2), using 3 N NaOH solution.
2. The composition formulations refer to Table 27. The thermal treatment A was performed under high temperature and normal pressure (60° C., 1.0 atm), while the thermal treatment B was performed under high temperature and high pressure (121° C., 1.5 atm).
3. For the supernatants with similar turbidity, when the settled sludge volume (SSV) is smaller, the sludge settling speed is faster and the sludge concentration is higher.

TABLE 30
The influence of the different nitrogen sources in the formulations on the
flocculating capacities

| | | | pH = 5.8, | | | | |
|---|---|---|---|---|---|---|---|
| Flocculant type and dosage | | | SSV (mL/500 mL) | | | | PH = 6.5 |
| Type | Thermal treatment | Dosage | 2 min | 3 min | 5 min | 40 min | Floc |

| | | | | |
|---|---|---|---|---|
| Control | 450 | 310 | 250 | 92 |
| Example | | | | |

| | | | | | |
|---|---|---|---|---|---|
| Formulation 13-1 | B | 0.5 mL/L | 260 | 170 | 150 |
| Formulation 13-2 | B | 0.5 mL/L | 250 | 170 | 150 |
| Formulation 13-3 | B | 0.5 mL/L | 250 | 170 | 140 |
| Formulation 13-4 | B | 0.5 mL/L | 250 | 160 | 140 |

Note:
1. The testing solution was prepared by adding 300 mg/L PACI into the textile dyeing wastewater (Lang-Bun), and then adjusting the pH values to 5.8 or 6.5 using 3 N NaOH solution. This was the control.
2. The composition formulations refer to formulation 13 in Table 27. The nitrogen source in formulation 13-1 was MP-90, the nitrogen source in formulation 13-2 was EG-90, the nitrogen source in formulation 13-3 was HI-90, and the nitrogen source in formulation 13-4 was Supro-620.
3. The thermal treatment A was performed under high temperature and normal pressure (60° C., 1.0 atm), while the thermal treatment B was performed under high temperature and high pressure (121° C., 1.5 atm).
4. For the supernatants with similar turbidity, when the settled sludge volume (SSV) is smaller, the sludge settling speed is faster and the sludge concentration is higher.

### Example 19

### Tuning Formulations of Biological Flocculants

The production culture formulation was further fine tuned with respect to nitrogen sources. The types of the nitrogen sources in the production formulations had no obvious effects on the flocculating activities, but the concentrations of the nitrogen sources can influence the flocculating activities. After thermal treatment, formulations containing high concentration nitrogen sources turned into gels and impeded the mechanical operation of the fermentation tank. Therefore, formulations needed fine-tuning to adjust the concentration of the nitrogen sources, suitable for the mechanical operation of the fermentation tank. Based on formulation no. 14 in Table 27, the influence of nitrogen source concentrations varying from 50 g/L to 100 g/L on the flocculating activities was studied. The experimental results are listed in Table 31, showing that higher nitrogen source concentrations promoted the early-stage settling speed of the flocs. However, after settling for 10 minutes, no noticeable enhancing effects were observed.

The nitrogen source in high concentrations contributed little to the flocculating activities but had negative influences on the mechanical operation of the fermentation tank. Due to the difficulty in uniformly heating the formulations containing high concentrations of nitrogen sources, the sterilization of the formulations was often incomplete, leading to easily degradable biological flocculants. It was decided that soybean protein with a concentration of 100 g/L is the appropriate basis for all the production formulations. Soybean protein was chosen for the reason that it is easy to obtain and has a low cost.

TABLE 31
The influence of different concentrations of nitrogen
source on the flocculating activities

| Flocculant type and dosage | | | SSV (mL/500 mL) | | | | |
|---|---|---|---|---|---|---|---|
| | Thermal | | | | | 10 | 40 |
| Type | treatment | Dosage | 2 min | 3 min | 5 min | min | min |
| Control | | | 460 | 400 | 270 | 150 | 70 |
| Example | | | | | | | |
| 50 g/L | A | 0.5 mL/L | 350 | 270 | 175 | 115 | 60 |
| 75 g/L | A | 0.5 mL/L | 330 | 260 | 160 | 105 | 60 |
| 100 g/L | A | 0.5 mL/L | 190 | 150 | 108 | 80 | 56 |
| 100 g/L | A | 0.5 mL/L | 210 | 155 | 125 | 98 | 60 |

Note:
1. The testing solution was prepared by adding 300 mg/L PACI into the textile dyeing wastewater (Lang-Bun), and then adjusting the pH values to 6.62 or 6.5 using 3 N NaOH solution. This was the control.
2. The composition formulations refer to formulation 14 in Table 27. The nitrogen source was Supro-620.
3. The thermal treatment A was performed under high temperature and normal pressure (60° C., 1.0 atm).
4. For the supernatants with similar turbidity, when the settled sludge volume (SSV) is smaller, the sludge settling speed is faster and the sludge concentration is higher.

### Example 20

### Thermal Treatment Conditions

The effect of thermal treatment in the preparation of biological flocculants was studied. Because the viscosity characteristics of the production formulations can cause changes to the normal operation of the fermentation tank, three types of thermal treatments were used. These three thermal treatments, high temperature and normal pressure (60° C., 1 atm, 40 minutes, BioFloc-HS), high temperature and high pressure (121° C., 1.5 atm, 20 minutes, BioFloc-AS) and room temperature dissolution (BioFloc-NS) were used to investigate the influence of thermal treatments on the viscosity of the production formulations.

The results for the flocculating activities of the biological flocculants prepared by the three types of thermal treatments toward the textile dyeing wastewater (Shin-Long) are listed in Table 32. The control (treating only with aluminum chloride solution) can eliminate 75% of the chemical oxygen demand in the wastewater, but fails to show evident removal effects for the biological oxygen demand in the wastewater. After adding the chemical flocculants or the biological flocculants and settling for 40 minutes, the settled sludge volume and the chemical oxygen demand of the supernatants were not significantly reduced, while the biological oxygen demand was reduced 50% or more if compared with the control.

Biological oxygen demand (BOD, unit: mg/L) is used to evaluate the content of organic compounds that will be degraded by the microbes in an oxygen-consuming manner, in the water. BOD is expressed as the consumed dissolved oxygen amount (DO, unit: mg/L) for degrading the pollutants by the microbes in the water. The method for measuring BOD refers to the analysis method “The measuring method for BOD of the water—NIEA W510.50A” published by the Environmental Protection Administration, Taiwan, R.O.C. After an appropriate amount of water sample is incubated in the dark under 20° C. for 5 days, the change (reduction) in the dissolved oxygen amount for the water sample in the experimental container is measured and shown as BOD₅.

The chemical flocculants had no obvious effects toward the suspended particle concentration of the supernatants, while the biological flocculants increases about 30% of the suspended particle concentration of the solutions.

The method for determining the total solid content (unit: mg/L) and total suspended solid content (unit: mg/L) of the water sample refers to the analysis method “The measuring method for the total solid content and total suspended solid content of the water—NIEA W210.55A” published by the Environmental Protection Administration, Taiwan, R.O.C. The measurement of the total solid content in the water is to dry-up the water sample in an 103° C.˜105° C. oven until reaching the constant weight and obtain the total solid content in the water sample by measuring the remained solid weight. The measurement of the total suspended solid content in the water is to filter the water sample through glass fiber filter, dry-up the filter in the 103° C.˜105° C. oven until reaching the constant weight and obtain the total suspended solid content in the water sample by calculating the increased weight of the filter. The total dissolved solid content is obtained from deducing the total suspended solid content from the total solid content.

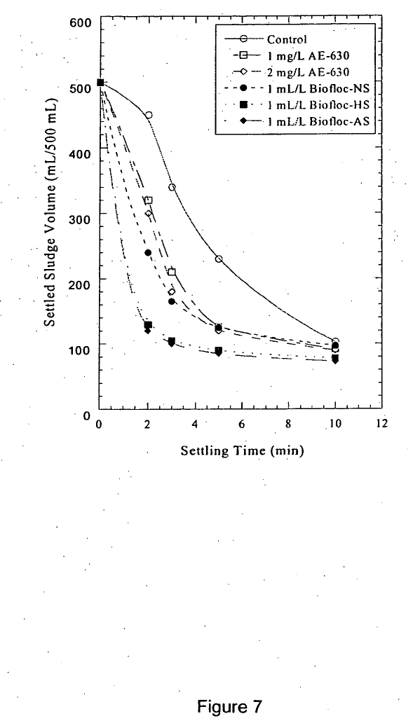

In general, when the density of the floc was higher, the settling speed was faster and the required settling time was shorter, thus increasing the tolerance for the flow changes in the clarifiers. FIG. 7 shows the changes in the settled sludge volume during the first ten minutes of settling. After settling for more than 10 minutes, the settled sludge volume was not correlated with the treatment methods. However, for the settling time less than 10 minutes, the samples treated by the biological flocculants, especially BioFloc-HS and BioFloc-AS, were observed with less settled sludge volume, representing a faster settling speed for the sludge. The wastewater treated by BioFloc-HS and BioFloc-AS had higher tolerance for the flow changes.

TABLE 32
The influence of the preparation conditions on the flocculating capacities

| | | SSV (mL/500 mL) | | | | | COD | SS | BOD |
|---|---|---|---|---|---|---|---|---|---|
| Flocculant type | Dosage | 2 min | 3 min | 5 min | 10 min | 40 min | (mg/L) | (g/L) | (mg/L) |
| Untreated wastewater | | | | | | | 1760 | | 160 (54) |
| Control | | 360 | 240 | 130 | 92 | 58 | 439 | 1.17 | 178 (72) |
| Chemical example | | | | | | | | | |
| EA-630 | 1 mg/L | 320 | 210 | 126 | 92 | 55 | 388 | 1.28 | 120 |
| EA-630 | 2 mg/L | 300 | 180 | 120 | 90 | 53 | 377 | 0.93 | 87 |
| Biological example | | | | | | | | | |
| BioFloc-NS | 1 mL/L | 240 | 165 | 125 | 97 | 62 | 348 | 1.74 | 78 |
| BioFloc-HS | 1 mL/L | 130 | 105 | 90 | 78 | 55 | 328 | 1.50 | 78 |
| BioFloc-AS | 1 mL/L | 120 | 100 | 85 | 73 | 55 | 387 | 1.65 | 81 |

Note:
1. The biological flocculant BioFloc-HS was prepared by high temperature and normal pressure (60° C., 1 atm, 40 minutes), the biological flocculant BioFloc-AS was prepared by high temperature and high pressure (121° C., 1.5 atm, 20 minutes) and the biological flocculant BioFloc-NS was prepared by normal temperature and normal pressure (room temperature dissolution).
2. SSV is the settled sludge volume during a specific settling time.
3. The testing solution was prepared by adding 300 mg/L PACI into the textile dyeing wastewater (Shin-Long), and then adjusting the pH value to 6.57 using 3 N NaOH solution. This was the control.
4. The composition formulations refer to formulation 13 in Table 27. The nitrogen source was Supro-620.
5. The value in the biological oxygen demand ( ) is the BOD value measured after filtering the sample with 0.45 μm membrane.
6. For the supernatants with similar turbidity, when the settled sludge volume (SSV) is smaller, the sludge settling speed is faster and the sludge concentration is higher.

### Example 21

### Influence of Drying Treatments on Flocculating Activity

The effect of spray drying the biological flocculant to form powders was studied in this experiment. It has been proved, as shown supra, that the flocculating activities of the liquid biological flocculants are comparable to those of chemical flocculants. Moreover, the biological flocculants can be used to treat the wastewater from the textile dyeing industry, without negative influences on the quality of the effluents. However, the biological liquid products are prone to contamination, thus deteriorating the activity and shortening the shelf life. Therefore, the biological flocculant products are produced in the powder form in order to achieve prolonged reservation.

This study used the spray dryer (EYELA, Spray Dryer SD-1) to perform the spray drying tests of the biological flocculants. The operation conditions of the spray dryer included setting the inlet and outlet temperatures of the spray dryer to 110° C. and 90° C. respectively. The powders obtained from spray drying were mixed with tap water to form a colloidal solution of 100 g/L. The flocculating activities of the biological flocculant and the biological flocculant treated with drying treatments toward the textile dyeing wastewater were tested and the results are listed in Table 33. According to the results of the settled sludge volume, the supernatant absorbance (OD₅₅₀) and the supernatant COD value, the biological flocculants that was treated by the spray drying and then mixed with the tap water had the flocculating capabilities equivalent to the biological flocculant without spray drying treatment.

TABLE 33
The influence of the drying treatment on the flocculating capacities.

| | | Settled Sludge Volume (mL/500 mL) | | | | | | |
|---|---|---|---|---|---|---|---|---|
| Flocculant type | Dosage | 2 min | 3 min | 5 min | 10 min | 40 min | OD₅₅₀ | COD (mg/L) |
| Untreated wastewater | | | | | | | 0.32 | 1025 |
| Control | | 400 | 350 | 250 | 142 | 85 | 0.073 | 466 |
| EA-630 | 5 mg/L | 200 | 140 | 106 | 76 | 52 | 0.044 | 434 |
| BioFloc-AS, Spray Dry | 50 mg/L | 205 | 160 | 125 | 95 | 68 | 0.064 | 468 |
| BioFloc-AS, Spray Dry | 20 mg/L | 215 | 165 | 130 | 98 | 70 | 0.046 | 434 |
| BioFloc-AS | 0.5 mL/L | 200 | 158 | 120 | 92 | 68 | 0.066 | 475 |
| BioFloc-AS | 0.2 mL/L | 210 | 165 | 130 | 98 | 70 | 0.045 | 441 |

Note:
1. The biological flocculant BioFloc-AS was prepared by high temperature and high pressure (121° C., 1.5 atm, 20 minutes) and the production formulation refers to the formulation 13 in Table 27, the nitrogen source is Supro-620.
2. For the supernatants with similar turbidity, as the settled sludge volume (SSV) is smaller, the sludge settling speed is faster and the sludge concentration is higher.
3. The testing solution was prepared by adding 450 mg/L PACI into the textile dyeing wastewater (Hor-Yo), and then adjusting the pH value to 6.3. using 3 N NaOH solution. This was the control.

### Example 22

### Influence of Viscosity on Flocculating Activity

The viscosity of biological flocculant was studied. As chemical flocculant contains more effective ingredients and has stronger flocculating activities, the viscosity of the chemical flocculant solution is higher. This experiment intends to investigate whether the biological flocculant has the character of better flocculating activities along with higher viscosity. Thus, the variation in viscosity can be used as an index for the quality of the biological flocculant product.

The changes in viscosity of the biological flocculant and the flocculating activity equivalence of the biological flocculant to the chemical flocculant are listed in Table 34. Based on the floc settling situations for the coagulation treatment of the textile dyeing wastewater (Shin-Long), the amount added (dosage) for the chemical flocculant EA-630, which has the same varying trend in the settled sludge volume, was used as the equivalent amount of chemical flocculant to biological flocculant. When the chemical flocculant equivalent amount is higher, it means the biological flocculant has better flocculating activities. The viscosity of the biological flocculant increased as the thermal treatment time increased, and the viscosity reached the peak at the thermal treatment time of 40 minutes. When the thermal treatment time was extended to 60 minutes, the viscosity started decreasing. Based on an analysis of the various chemical flocculant equivalent amounts, no direct relationship was observed between the thermal treatment time, the viscosity and the flocculating activities. However, higher temperatures of the thermal treatment contributed a lot to the flocculating activities. The production formulation (BioFloc-AS) treated under high temperature and high pressure had a viscosity of 70 cpc and chemical flocculant equivalent of 8 mg/L of EA-630.

The viscosity tests for the samples were examined by vertical roller viscometer (BROOKFIELD MODEL-DV-11, USA), the steps included: selecting the rotator with the suitable viscosity (the higher viscosity with the smaller size), pouring 25-30 mL sample to the outer roller, starting the rotator and adjusting the rotating speed, and finally acquiring the viscosity value.

TABLE 34
The influence of the thermal treatment time on the flocculating activities.

| | | | Equivalence of 1 mg/L to | |
|---|---|---|---|---|
| Biological | | Treatment | chemical flocculant EA- | Viscosity |
| flocculant | Thermal treatment | time | 630 | (cps) |
| BioFloc- | high temperature and high pressure | 20 min | 8 mg/L EA-630 | 70 |
| AS | | | | |
| BioFloc- | room temperature dissolution | 0 min | 2 mg/L EA-630 | |
| NS | | | | |
| BioFloc- | high temperature and normal | 20 min | 5 mg/L EA-630 | 845 |
| HS | pressure | | | |
| BioFloc- | high temperature and normal | 30 min | 5 mg/L EA-630 | 1980 |
| HS | pressure | | | |
| BioFloc- | high temperature and normal | 40 min | 5 mg/L EA-630 | 2020 |
| HS | pressure | | | |
| BioFloc- | high temperature and normal | 60 min | 5 mg/L EA-630 | 1850 |
| HS | pressure | | | |

Note:
1. The biological flocculant BioFloc-HS was prepared by high temperature and normal pressure (60° C., 1 atm, 40 minutes), the biological flocculant BioFloc-AS was prepared by high temperature and high pressure (121° C., 1.5 atm, 20 minutes) and the biological flocculant BioFloc-NS was prepared by normal temperature and normal pressure (room temperature dissolution).
2. The testing solution was prepared by adding 300 mg/L PACI into the textile dyeing wastewater (Shin-Long), and then adjusting the pH value to 6.57 using 3 N NaOH solution. This was the control.
3. Based on the floc settling situations for the coagulation treatment of the textile dyeing wastewater, the addition amount of the chemical flocculant (EA-630) with the same varying trend in the settled sludge, volume was used as the equivalent amount of chemical flocculant to biological flocculant. When the equivalence is higher, it means the biological flocculant has better flocculating activities.

### Example 23

### Biological Inhibition Analysis of Biological Flocculant

A study was performed to analyze the toxicity of biological flocculant using MicroTox® analyzer. Biological toxicity is expressed as biological inhibition which is analyzed by MicroTox® by way of the luminescent bacteria (Vibro flscheri, Photobacterium phosphoreum, NRRL No. B-11177) isolated from the sea water, of which the luminescent intensity is decreased/inhibited by the toxic materials in the environment, to examine the biological toxicity of the sample. The MicroTox® analyzer uses the highly sensitive photomultiplier to express the luminosity before and after the microbes are exposed to the toxic materials, in values. Based on the principle that the luminosity intensity of the luminescent bacteria is inversely proportional to the concentration of the toxic material, the microbial activity inhibition becomes more evident by the showing of weaker luminosity when exposed to the more toxic material. In addition, the pH value should be kept neutral (pH=6˜8) during the experiment and the temperature is calibrated to 15° C. by the internal thermostat for minimizing the experimental errors as the temperature and the pH values may influence the luminescent activity of the microbes. When the luminescent microbes are added into the test sample, their luminescent ability is repressed. As the microbial fluorescent intensity is reduced to 50% of the initial intensity, the concentration of the toxic material at this moment is defined as EC₅₀. The biological toxicity tested by using MicroTox® biological toxicity tests is designated as EC₅₀ (t,T), while “t” represents the reaction time of the sample and the microbes and “T” represents the reaction temperature. Generally, the toxicity can be expressed by EC₅₀ (5 min, 15° C.) and EC₅₀ (15 min, 15° C.), despite that low toxicity can be expressed by EC₅₀ (30 min, 15° C.).

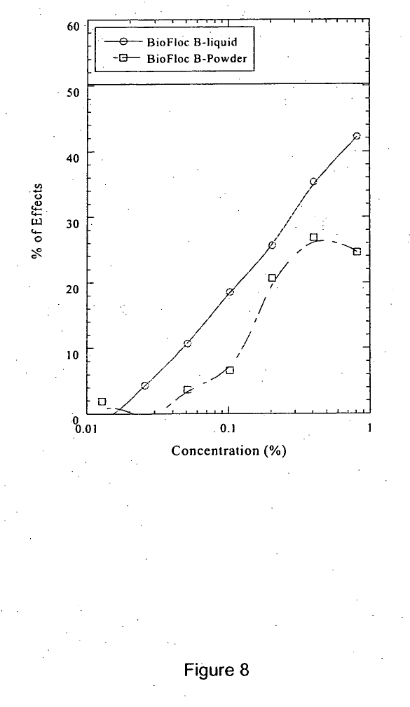

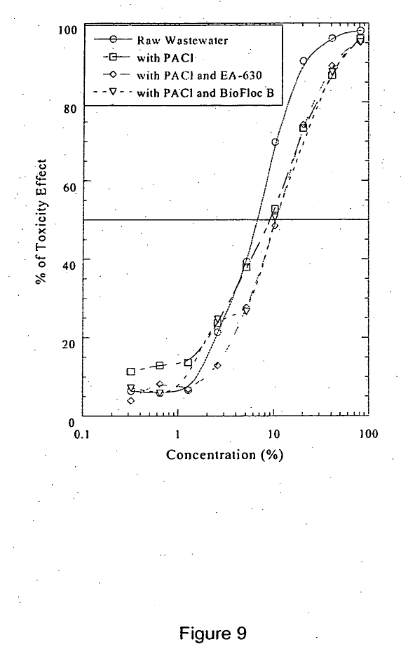

The biological: inhibition of BioFloc B was analyzed. BioFloc B is based on formulation 13 in Table 27 as the production formulation of the biological flocculant, using the nitrogen source of 100 g/L soybean protein, under thermal treatment at 121° C., 1.5 atm for 20 minutes. From various test results of the flocculants, the application ratio of the biological flocculant to the textile dyeing wastewater is generally below 1/1000. This experiment used 1% concentration to evaluate the toxicity of the biological flocculant. This concentration was about 10 times higher than the amounts commonly used. MicroTox® biological toxicity tests was used to analyze the biological inhibition of the biological flocculant. From the tests, no noticeable biological inhibition was observed for either the biological flocculant BioFloc B in the liquid form or in the powder form, see FIG. 8. The textile dyeing wastewater treated by the biological flocculant showed a slightly lower biological inhibition, when compared with the sample treated by the chemical flocculant (EA-630, 2 mg/L), see FIG. 9.

### Example 24

### Influence of Biological Flocculant Dosages on Flocculating Capability

In this experiment, the effect of different dosages of biological flocculant was studied. Using the textile dyeing wastewater, the flocculating activities of BioFloc A (B. endophyti fermentation solution), BioFloc B and the chemical flocculent (EA-630) 2 mg/L were compared and the results are listed in Table 35. The usage of the flocculant clearly enhanced the performances of coagulation treatment. With respect to the settled sludge volume, BioFloc A, BioFloc B and the chemical flocculent EA-630 had similar flocculating capabilities. From the results of the supernatant COD and the suspended particle concentration, BioFloc B showed better results in treating wastewater when compared to EA-630 and BioFloc A.

Influence of excess biological flocculant on the water quality was examined. For operations in the factories, over-dosage of coagulants often occurs due to variations in the water quality of the wastewater. When the chemical flocculent was used in excess, no apparent negative effects were observed for the settled sludge volume and the water quality of the supernatant. On the other hand, if the biological flocculant was used in excess, lower flocculating capabilities were observed as shown in Table 35. Based on two results of the coagulation tests, the most suitable dosage for BioFloc B was 0.2 mL/L, leading to an average COD value of 348 mg/L. When using the biological flocculant in excess (dosage 1.0 mL/L), the settled sludge volume was slightly reduced with the average COD value for the supernatant being 415 mg/L.

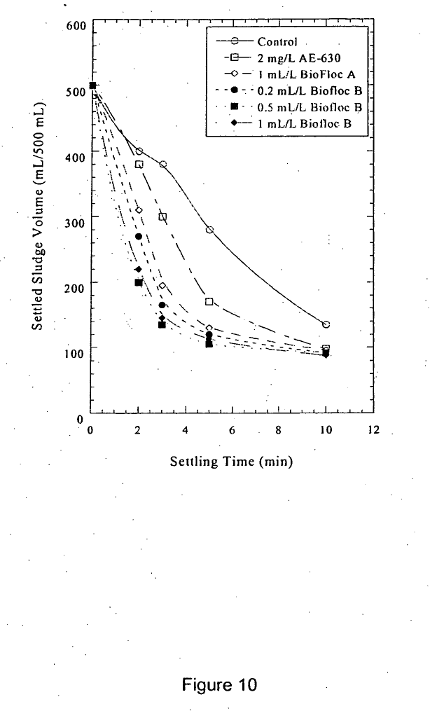

FIG. 10 shows the changes in the settled sludge volume during different settling time. In the early stage of settling, the flocs in the wastewater treated by biological flocculants showed faster settling speeds than those treated by the chemical flocculant, resulting in less settled sludge volume. After settling for more than 10 minutes, no obvious difference was observed for the settled sludge volume. When the biological flocculant BioFloc B was used in excess (dosage 1.0 mL/L), the settlement of the flocs was better with a faster settling speed in the early stage.

Using the textile dyeing wastewater (Shin-Long) as the testing solution, the analysis results of the chemical flocculantEA-630 and the biological flocculent BioFloc B are listed in Table 36. From the results, overdose of the biological flocculant showed no obvious negative effects on the water qualities for the supernatant, including the chemical oxygen demand (COD), the suspended solid concentration (SS), the color and the electrical conductivity, and had the treating effects comparable to the chemical flocculant. As the absorbance (OD₅₅₀) was increased, overdose of the biological flocculant led to the slightly lower flocculating activity, but still comparable to the chemical flocculant.

The electrical conductivity is the reverse of the electrical resistance of the electrical current passing through a liquid pillar with the cross-section of 1 cm² and the height of 1 cm, in the unit of mho/cm. If the conductivity is diminutive, it is expressed in the grade of 10⁻³ or 10⁻⁶ in the unit of mmho/cm or μmho/cm. The electrical conductivity is increased as the concentration of the dissolved ions in the water increases. The method for measuring the conductivity refers to the analysis method “The measuring method for water conductivity—NIEA W203.51 B” published by the Environmental Protection Administration, Taiwan, R.O.C., using the ohmmeter (ATI Orion, Model 130) calibrated by the standard conductivity solution.

Based on the flocculating activities of the biological flocculant (BioFloc B) and the chemical flocculant (EA-630) toward the textile dyeing wastewater (Shin-Long), the equivalent amount of EA-630 to BioFloc B was determined. The experimental results are listed in Table 37. When the chemical flocculant EA-630 was below the dosage of 10 mg/L, higher dosage led to better flocculating effect, faster settling speed of the flocs, and smaller settled sludge-volume. As for the treating effects of the biological flocculant BioFloc B toward the same wastewater, except for the supernatant COD, the properties of the settled sludge and other water qualities were superior.

TABLE 35
The evaluation of the addition amount of the biological flocculant toward
the untreated wastewater from the Shin-Long factory

| | | Settled Sludge Volume (mL/500 mL) | | | | | | |
|---|---|---|---|---|---|---|---|---|
| Flocculant type | Dosage | 2 min | 3 min | 5 min | 10 min | 40 min | COD (mg/L) | SS (g/L) |
| Untreated wastewater | | | | | | | 1430 | |
| Control 1 | | 460 | 400 | 250 | | 45 | 529 | 1.09 |
| Chemical example 1 | | | | | | | | |
| EA-630 | 2 mg/L | 420 | 300 | 120 | | 42 | 533 | 1.23 |
| Biological example 1 | | | | | | | | |
| BioFloc A | 1 mL/L | 390 | 275 | 125 | | 50 | 518 | 0.32 |
| BioFloc B | 0.2 mL/L | 350 | 260 | 130 | | 56 | 387 | 0.15 |
| BioFloc B | 0.5 mL/L | 250 | 130 | 100 | | 50 | 392 | 0.93 |
| BioFloc B | 1 mL/L | 200 | 125 | 98 | | 50 | 452 | 0.21 |
| Control 2 | | 450 | 380 | 280 | 135 | 70 | 334 | 0.90 |
| Chemical example 2 | | | | | | | | |
| EA-630 | 2 mL/L | 380 | 300 | 170 | 98 | 48 | 302 | 0.64 |
| Biological example 2 | | | | | | | | |
| BioFloc A | 1 mL/L | 310 | 195 | 130 | 96 | 56 | 389 | 0.90 |
| BioFloc B | 0.2 mL/L | 270 | 165 | 120 | 92 | 56 | 308 | 0.73 |
| BioFloc B | 0.5 mL/L | 200 | 135 | 105 | 92 | 50 | 331 | 0.76 |
| BioFloc B | 1 mL/L | 220 | 145 | 113 | 88 | 51 | 377 | 0.44 |

Note:
1. the biological flocculant BioFloc A is the fermentation solution of the microbe B. endophyti, BioFloc B is the culture medium prepared under high temperature and high pressure (121° C., 1.5 atm).
2. The testing solution was prepared by adding 350 mg/L PACI into the textile dyeing wastewater (Shin-Long), and then adjusting the pH value to 6.3 using 3 N NaOH solution. This was the control.
3. For the supernatants with similar turbidity, as the settled sludge volume (SSV) is smaller, the sludge settling speed is faster and the sludge concentration is higher.

TABLE 36
The influence of the addition amount of the biological flocculant on the
flocculating capabilities

| | | Settling 5 min | | COD | SS | Color | Conductivity |
|---|---|---|---|---|---|---|---|
| Flocculant type | Dosage | OD₅₅₀ | Activity | (mg/L) | (mg/L) | (ADMI) | (μs/cm) |
| Untreated wastewater | | 0.318 | | 1410 | | | 1418 |
| Control | | 0.092 | 7.7 | 414 | 12 | 74 | 1824 |
| EA-630 | 5 mg/L | 0.034 | 26.3 | 383 | 12 | 75 | 1816 |
| BioFloc B | 0.05 mL/L | 0.027 | 33.9 | 383 | 14 | 78 | 1824 |
| BioFloc B | 0.1 mL/L | 0.028 | 32.6 | 394 | 16 | 74 | 1823 |
| BioFloc B | 0.2 mL/L | 0.034 | 26.3 | 391 | 17 | 78 | 1825 |
| BioFloc B | 0.5 mL/L | 0.026 | 35.3 | 401 | 6 | 75 | 1824 |

Note:
1. The testing solution was prepared by adding 500 mg/L PACI into the textile dyeing wastewater (Shin-Long), and then adjusting the pH value to 6.45 using 3 N NaOH solution. This was the control.
2. Flocculating Activity = 1/OD_{550,S} − 1/OD_{550,C} OD_{550,S} = optical density of the sample under wavelength 550 nm OD_{550,C} = optical density of the control under wavelength 550 nm
3. For the supernatants with similar turbidity, as the settled sludge volume (SSV) is smaller, the sludge settling speed is faster and the sludge concentration is higher.

TABLE 37
The influence of the addition amount of the chemical organic flocculant on
the flocculating capabilities

| | | SSV (mL/500 mL) | | | | COD | | Color | SS |
|---|---|---|---|---|---|---|---|---|---|
| Flocculant type | Dosage | 2 min | 3 min | 5 min | 10 min | (mg/L) | OD₅₅₀ | (ADMI) | (mg/L) |
| Control | | 450 | 310 | 95 | 50 | 1060 | 0.255 | 96 | 94 |
| Biological example | | | | | | | | | |
| BioFloc B | 0.5 mL/L | 90 | 89 | 65 | 50 | 393 | 0.072 | 71 | 70 |
| Chemical example | | | | | | | | | |
| EA-630 | 2 mg/L | 330 | 215 | 85 | 55 | 281 | 0.178 | 85 | 95 |
| EA-630 | 3 mg/L | | | | | | | | |
| EA-630 | 5 mg/L | 210 | 110 | 70 | 50 | 323 | 0.156 | 75 | 90 |
| EA-630 | 10 mg/L | 80 | 60 | 50 | 30 | 320, 344 | 0.137 | 72 | 70 |

Note:
1. The testing solution was prepared by adding 350 mg/L PACI into the textile dyeing wastewater (Shin-Long), and then adjusting the pH value to 6.52 using 3 N NaOH solution. This was the control.
2. For the supernatants with similar turbidity, as the settled sludge volume (SSV) is smaller, the sludge settling speed is faster and the sludge concentration is higher.

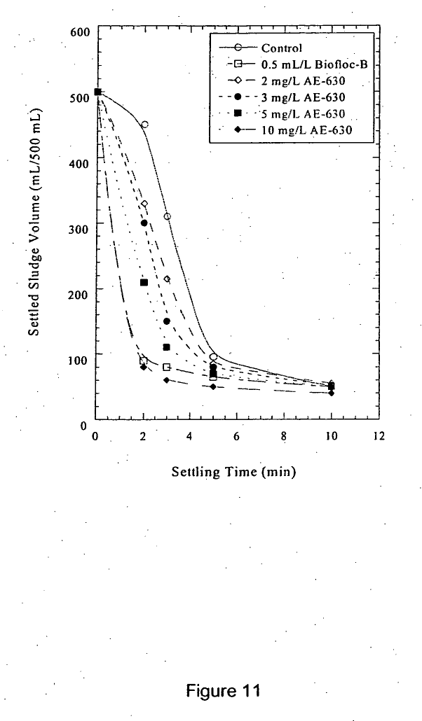

FIG. 11 shows the changes in the settled sludge volume in different settling time. In the early stage of settling, as more chemical flocculent was present, the sludge settled better. However, the flocs treated by biological flocculants showed slower settling speed than that of the chemical flocculant EA-630 in the dosage of 10 mg/L. In general, although the addition of the flocculent, in addition to the coagulant, showed no obvious contribution to the water qualities for the supernatant, including. COD, SS, the color and the conductivity, the settling speeds of the flocs were faster than those treated by only coagulants (aluminum chloride).

### Example 25

### Influence of Cation Coagulants on Flocculating Capability of Biological Flocculant

The effect of a cation coagulant in combination with biological flocculant was studied. During the coagulation treatment of the industrial wastewater, it is common to use a coagulant and a flocculant at the same time to enhance the treating effects. However, due to the differences in the suspended solid particle properties in the water and the water quality of the industrial wastewater, different coagulants may have an effect on the flocculating activities of the flocculant. This experiment mainly assessed the influences of the common cation coagulants, for example, ferrous sulfate (FeSO₄), magnesium sulfate (MgSO₄), calcium chloride (CaCl₂), ferric chloride (FeCl₃) and aluminum chloride (AlCl₃) etc., on the flocculating capabilities of the biological flocculant BioFloc B. Using the textile dyeing wastewater (Shin-Long) as the testing solution, changes in the flocculating activities of the biological flocculant, in combination with different cation coagulants, are listed in Table 38. The results indicate that the coagulation treatment procedure using the biological flocculant with aluminum chloride has the best treating effect on the textile dyeing wastewater.

TABLE 38
The influence of the cation coagulants on the flocculating capabilities of the biological flocculant

| Coagulant | Dosage | PH | |
|---|---|---|---|
| type | (mg/L) | range | Flocculating effects |
| AlCl₃ | 350 | 6.5 | Complete settling in about 10 minutes |
| CaCl₂ | 300˜1000 | 5˜8 | With the salt dosage between 300 mg/L to 1000 mg/L, the operation |
| | | | pH range between 5-8, no setting efffects were observed. |
| FeSO₄ | 400˜800 | 5˜8 | With the salt dosage between 400 mg/L to 800 mg/L, the operation |
| | | | pH range between 5-8, settling effects are observed, but the |
| | | | supernatant had dark color. |
| MgSO₄ | 400˜1000 | 5˜8 | With the salt dosage between 400 mg/L to 1000 mg/L, the operation |
| | | | pH range between 5-8, no settling effects were observed |
| FeCl₃ | 300˜500 | 6˜8 | With the salt dosage between 300 mg/L, to 500 mg/L, the operation |
| | | | pH range between 6-8, higher the coagulant dosage is, more |
| | | | sediments is present. However, for the dosage up to 500 mg/L, |
| | | | suspended particles are observed in the supernatant. The most |
| | | | suitable operation pH is neutral. |

Note:
the testing solution was the textile dyeing wastewater (Shin-Long).

Using the tofu production wastewater (Heng-E) to test the influence of the cation coagulants, including magnesium sulfate, ferric chloride and aluminum chloride etc., on the coagulation treatment, the preliminary results (not shown) point out that ferric chloride and aluminum chloride had better flocculating activities. The results of using the coagulants ferric chloride and aluminum chloride combined with the flocculant for treating the tofu fabrication wastewater (Heng-E) are shown in Table 39. When the dosage of the aluminum salt or the ferric salt was 150 mg/L, the floc settling speeds of these two coagulation treatments were comparable, and the COD value of the supernatant was reduced from 1230 mg/L to about 300 mg/L. With respect to the absorbance (OD₅₅₀), aluminum chloride had better treating effects. The absorbance of ferric chloride was increased probably due to the incomplete removal of the ferric chloride particles from the supernatant.

Using the tofu production wastewater (Heng-E) as the testing solution, the influences of the dosages of the same inorganic cation coagulant on the flocculating capabilities of the biological flocculant and the chemical flocculant are shown in Table 39. Considering using the coagulants ferric chloride or aluminum chloride combined with the biological flocculant for treating the tofu production wastewater, no obvious difference was shown between these two coagulants. However, the flocs obtained from the combination of the chemical flocculant (EA-630) and aluminum chloride had slower settling speeds than the combination of ferric chloride and the chemical flocculant. It was probably because that the dosage of the ferric salt was higher than that of the aluminum salt, resulting in the floc density for the ferric salt larger than the aluminum salt and thus a faster settling speed.

As concluded from the above experimental results, the biological flocculant can be combined with the inorganic cation coagulants of ferric salts or aluminum salts for treating the industrial wastewater or the tofu production wastewater. Moreover, no obvious differences were observed between the water qualities after coagulation treatments of these two coagulants.

TABLE 39
The influence of coagulants aluminum chloride and ferric chloride on the
flocculating capabilities of the biological flocculant

| | | Settled Sludge Volume (mL/500 mL) | | | | | | COD | |
|---|---|---|---|---|---|---|---|---|---|
| Coagulant type | Dosage | 1 min | 2 min | 3 min | 5 min | 10 min | 40 min | (mg/L) | OD₅₅₀ |
| Untreated | | | | | | | | 1230 | |
| wastewater | | | | | | | | | |

AlCl₃ 150 mg/L, pH = 6.50

| | | | | | | | |
|---|---|---|---|---|---|---|---|
| Control | | 230 | 150 | 130 | 100 | 293 | 0.072 |
| EA-630 | 5 mg/L | 220 | 150 | 130 | 98 | 271 | 0.066 |
| BioFloc B | 0.2 mL/L | 200 | 135 | 120 | 95 | 296 | 0.054 |

FeCl₃ 150 mg/L, pH = 6.50

| | | | | | | | |
|---|---|---|---|---|---|---|---|
| Control | | 270 | 190 | 160 | 115 | 286 | 0.086 |
| BioFloc B | 0.5 mL/L | 200 | 150 | 130 | 100 | 305 | 0.125 |
| BioFloc B | 1 mL/L | 200 | 150 | 130 | 100 | 333 | 0.111 |

FeCl₃ 300 mg/L, pH = 6.40

| | | | | | |
|---|---|---|---|---|---|
| Control | | 310 | 220 | 150 | 90 |
| BioFloc B | 0.2 mL/L | 240 | 175 | 130 | 90 |
| EA-630 | 5 mg/L | 220 | 160 | 110 | 80 |

AlCl₃ 250 mg/L, pH = 6.40

| | | | | | |
|---|---|---|---|---|---|
| Control | | 320 | 240 | 170 | 90 |
| BioFloc B | 0.2 mL/L | 250 | 190 | 140 | 90 |
| EA-630 | 5 mg/L | 340 | 250 | 170 | 92 |

Note:
1. The testing solution was the tofu production wastewater (Hong-E).
2. After adding FeCl3 or PACI, the pH value was adjusted bu using 3 N NaOH solution. This was the control.
3. For the supernatants with similar turbidity, as the settled sludge volume (SSV) is smaller, the sludge settling speed is faster and the sludge concentration is higher.

### Example 26

### Development of Flocculant

Flocculating activities of several solutions were examined in developing the best flocculating solution. The standard solution for testing the flocculating activities was prepared by adding 400 mg/L of aluminum chloride (PACl) coagulant into the textile dyeing wastewater, and then adjusting the pH values to 6.97. Fermented solutions with and without bacteria were prepared as set out in Tables 2 and 3. Using the jar tests to assess various fermented solutions, the settled sludge volume were taken and listed in Table 2, for wastewater from Wu-Li, and Table 3, for wastewater from Shin-Long. In table 2, comparing the settled sludge volume following settling for 2 minutes, the non-inoculated culture medium and the 48 hour fermentation solution had similar flocculating capabilities. The same situation can be observed in the flocculation tests of the textile dyeing wastewater from the other factory, Shin-Long, see Table 3.

TABLE 2
The evaluation of the flocculating capacities by the fermented bacterial
solution of the biological flocculant producing bacteria toward the wastewater
from textile dyeing industries (Wu-Li)

| | SSV (mL/500 mL) | | | | |
|---|---|---|---|---|---|
| Flocculant type and dosage | 2 min | 3 min | 5 min | 10 min | 40 min |
| Control | 440 | 355 | 155 | 105 | 45 |
| Example | | | | | |
| Adding 0 hr fermentation solution 1, 1 mL/L | 110 | 85 | 70 | 55 | 36 |
| Adding 24 hr fermentation solution 1, 1 mL/L | 250 | 122 | 88 | 72 | 36 |
| Adding 48 hr fermentation solution 1, 1 mL/L | 110 | 85 | 70 | 58 | 36 |
| Adding 0 hr fermentation solution 2, 1 mL/L | 210 | 145 | 95 | 70 | 37 |
| Adding 48 hr fermentation solution 2, 1 mL/L | 310 | 190 | 110 | 88 | 40 |

Note:
1. The testing solution was prepared by adding 400 mg/L PACI into the textile dyeing wastewater (Wu-Li), and then adjusting the pH values to 6.97 using 3 N NaOH solution. This was the control.
2. The composition of the culture medium included Supro 620 15 g/L, glucose 20 g/L, molasses 5 g/L and yeast extract 5 g/L.
3. For the supernatants with similar turbidity, when the settled sludge volume (SSV) is smaller, the sludge settling speed is faster and the sludge concentration is higher.

TABLE 3
The evaluation of the flocculating capacities by the fermented bacterial
solution of the biological flocculant producing bacteria toward the wastewater
from the textile dyeing industries (Shin-Long).

| | SSV (mL/500 mL) | | | | |
|---|---|---|---|---|---|
| Flocculant type and dosage | 2 min | 3 min | 5 min | 10 min | 40 min |
| Control | 450 | 350 | 185 | 100 | 60 |
| Example | | | | | |
| 1 mg/L EA-630 | 350 | 320 | 130 | 80 | 47 |
| Adding 0 hr fermentation solution 1, 1 mL/L | 280 | 185 | 120 | 86 | 55 |
| Adding 0 hr fermentation solution 2, 1 mL/L | 160 | 125 | 100 | 76 | 51 |
| Adding 0 hr fermentation solution 3, 1 mL/L | 160 | 130 | 100 | 77 | 51 |
| Adding 40 hr fermentation solution 1, 1 mL/L | 230 | 170 | 120 | 85 | 52 |
| Adding 40 hr fermentation solution 2, 1 mL/L | 200 | 150 | 110 | 80 | 52 |
| Adding 40 hr fermentation solution 3, 1 mL/L | 200 | 155 | 116 | 83 | 52 |

Note:
1. The testing solution was prepared by adding 350 mg/L PACI into the textile dyeing wastewater (Shin-Long), and then adjusting the pH values to 6.72 using 3 N NaOH solution. This was the control.
2. The composition of the culture medium included Supro 620 15 g/L, glucose 20 g/L, molasses 5 g/L and yeast extract 5 g/L.

### REFERENCES

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The references are also incorporated herein by reference.

1-57. (canceled)

58. A method of treating a quantity of water comprising:
obtaining a composition comprising soybean protein, wherein the composition is at least about 15 grams/liter soybean protein; and
adding the composition to the quantity of water, wherein at least a portion of particles in the quantity of water is flocculated and the quantity of water is treated.

59. The method of claim 58, wherein the water is industrial wastewater.

60. The method of claim 59, wherein the industrial wastewater is wastewater from an industry selected from a dyeing industry, a food industry, and a cosmetic industry.

61. The method of claim 58, wherein the composition increases the settling rate of suspended particles.

62. The method of claim 58, wherein the composition increases the volume of settled sludge.

63. The method of claim 58 wherein the composition has a concentration of soybean protein selected from about 15 grams/liter, 50 grams/liter, 75 grams/liter, 100 grams/liter, and 120 grams/liter.

64. The method of claim 58 wherein the soybean protein has been subjected to thermal treatments of about 60° C. to about 121° C. and about 1 atm. to about 1.5 atm. for about 20 minutes to about 40 minutes.

65. The method of claim 58 wherein the pH is adjusted to a range of about 5.8 to about 6.62.

66. The method of claim 58, wherein the composition is introduced in a dosage of about 0.2 ml/liter to about 1 ml/liter.

67. The method of claim 58, further comprising adding a cationic coagulant separately or in combination with the composition, wherein adding the cationic coagulant increases the settling rate of suspended particles and/or increases the volume of settled sludge.

68. The method of claim 67, wherein the cationic coagulant is selected from aluminum chloride, calcium chloride, ferric chloride, ferrous sulfate, and magnesium sulfate.
